(19) United States
(12) Patent Application Publication
(10) Pub. No.: US 2009/0162314 A1
(43) Pub. Date: Jun. 25, 2009

(54) Magnesium-Containing Polymers for the Treatment of Hyperphosphatemia

(76) Inventors: Chad C. Huval, Somerville, MA (US); Pradeep K. Dhal, Westford, MA (US); Stephen Randall Holmes-Farley, Arlington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/083,750

(22) PCT Filed: Nov. 7, 2006
(86) PCT No.: PCT/US2006/043402

§ 371 Date: May 30, 2008

Related U.S. Application Data
(60) Provisional application No. 60/734,593, filed on Nov. 8, 2005.

(51) Int. Cl. A61K 31/765; A61P 7/00

(52) U.S. Cl. 424/78.38

(57) ABSTRACT

A pharmaceutical composition comprising an aliphatic amine polymer or a pharmaceutically acceptable salt thereof, and a pharmaceutically acceptable magnesium compound comprising a magnesium ion is disclosed. A method of treating hyperphosphatemia in a patient is also disclosed. The method comprises the step of administering to the subject an effective amount of the disclosed pharmaceutical composition.

### RELATED APPLICATION

This application claims the benefit of U.S. Provisional Application No. 60/734,593, filed Nov. 8, 2005.

The entire teachings of the above application is incorporated herein by reference.

### BACKGROUND

People with inadequate renal function, hypoparathyroidism, or certain other medical conditions often have hyperphosphatemia, or elevated serum phosphate levels. Hyperphosphatemia, especially if present over extended periods of time, leads to severe abnormalities in calcium and phosphorus metabolism, often manifested by hyperparathyroidism, bone disease and calcification in joints, lungs, eyes and vasculature. For patients who exhibit renal insufficiency, elevation of serum phosphorus within the normal range has been associated with progression of renal failure and an increased risk of cardiovascular events.

Oral administration of certain phosphate binders such as magnesium compounds to treat elevated phosphate levels has been discussed. However, magnesium compounds may cause hypermagnesemia and osmotic diarrhea.

Polymer materials, such as aliphatic amine polymers, have also been used in the treatment of hyperphosphatemia. These polymers provide an effective treatment for decreasing the serum level of phosphate.

### SUMMARY

In one embodiment, the present invention is directed to a pharmaceutical composition comprising an aliphatic amine polymer or a pharmaceutically acceptable salt thereof, and a pharmaceutically acceptable magnesium compound comprising a magnesium ion, wherein the magnesium ion comprises 5-35% by anhydrous weight of the pharmaceutical composition.

In another embodiment, the present invention is directed to a pharmaceutical composition comprising an aliphatic amine polymer or a pharmaceutically acceptable salt thereof, and a pharmaceutically acceptable magnesium compound comprising a magnesium ion. The molar ratio of the magnesium ion to amine nitrogen atoms in the aliphatic amine polymer is 0.4-3.0

In yet another embodiment, the present invention is directed to a pharmaceutical composition comprising a crosslinked aliphatic amine polymer or a pharmaceutically acceptable salt thereof, and a pharmaceutically acceptable magnesium compound comprising a magnesium ion. The magnesium compound is entrained within the crosslinked aliphatic amine polymer.

In yet another embodiment, the present invention is directed to a pharmaceutical composition comprising an aliphatic amine polymer or a pharmaceutically acceptable salt thereof, and a pharmaceutically acceptable magnesium compound comprising a magnesium ion. The magnesium compound is selected from the group consisting of magnesium oxide, magnesium hydroxide, magnesium carbonate, magnesium formate, and a combination thereof.

The present invention also provides methods of treating a subject with hyperphosphatemia. The method comprises the step of administering to the subject an effective amount of a pharmaceutical composition disclosed herein.

### BRIEF DESCRIPTION OF THE DRAWINGS

FIG. 1 is a graph showing urinary magnesium excretion in Sprague Dawley (SD) rats treated with 0.5% diet of sevelamer hydrochloride.

FIG. 2 is a graph showing urinary magnesium excretion in Sprague Dawley (SD) rats treated with a pharmaceutical composition of the invention comprising crosslinked polyallylamine that includes a magnesium compound (PAA/Mg) in 0.25-0.5% low phosphate-diet.

FIG. 3 is a graph showing urinary magnesium excretion in Sprague Dawley (SD) rats treated with a pharmaceutical composition of the invention comprising crosslinked polyallylamine that includes a magnesium compound (PAA/Mg) in 2.6% high phosphate diet.

### DETAILED DESCRIPTION

As used herein, a “pharmaceutically acceptable magnesium compound” means a compound comprising a magnesium cation, which does not cause unacceptable side effects at the dosages which are being administered. The pharmaceutically acceptable magnesium compound can be water-soluble or water-insoluble.

It is to be understood that a “pharmaceutically acceptable magnesium compound” may encompass different polymorphs of the pharmaceutically acceptable magnesium compound. The term “polymorph” refers to solid crystalline forms of a compound, which may exhibit different physical, chemical or spectroscopic properties.

The “pharmaceutically acceptable magnesium compound” may also include various solvates of the pharmaceutically acceptable magnesium compound, which include a stoichiometric or non-stoichiometric amount of solvent, e.g., water or organic solvent, bound by non-covalent intermolecular forces.

Preferred pharmaceutically acceptable magnesium compounds have a high weight percentage of magnesium, and/or have a high density. These magnesium compounds can minimize daily dose volume. Examples of magnesium compounds suitable for the invention include magnesium oxide, magnesium hydroxide, magnesium halides (e.g., magnesium fluoride, magnesium chloride, magnesium bromide and magnesium iodide), magnesium alkoxides (e.g., magnesium ethoxide and magnesium isopropoxide), magnesium carbonate, magnesium bicarbonate, magnesium formate, magnesium acetate, magnesium trisilicates, magnesium salts of organic acids, such as fumaric acid, maleic acid, acrylic acid, methacrylic acid, itaconic acid and styrenesulfonic acid, and a combination thereof. When referring to any of these magnesium compounds, it is to be understood that mixtures, polymorphs and solvates thereof are encompassed.

Examples of preferred pharmaceutically acceptable magnesium compounds in the invention include magnesium oxide, magnesium hydroxide, magnesium carbonate and magnesium formate, and a combination thereof. Other examples of preferred magnesium compounds include magnesium bicarbonate, magnesium ethoxide and magnesium trisilicate. Magnesium oxide, magnesium hydroxide, or a mixture of magnesium oxide and magnesium hydroxide is more preferred in the invention.

In some embodiments, a pharmaceutically acceptable magnesium compound in the invention is not magnesium stearate or magnesium silicate.

Typically, the magnesium ion of the pharmaceutically acceptable magnesium compound comprises 5-35%, such as 10-30%, 10-25%, 13-25%, 15-22% and 16-20%, by anhydrous weight of the pharmaceutical composition.

Alternatively, the magnesium ion of the pharmaceutically acceptable magnesium compound comprises 5-35%, such as 10-30%, 10-25%, 13-25%, 15-22% and 16-20%, by anhydrous weight of the combined weight of the magnesium compound and the free base of the aliphatic amine polymer. Herein, the term “the free base of the aliphatic amine polymer” means the aliphatic amine polymer not including any counter ion. When the quantity of magnesium compound in the pharmaceutical composition is expressed in this fashion, it is to be understood that the aliphatic amine polymer in the pharmaceutical composition can be unprotonated, partially protonated or completely protonated. However, the weight of the aliphatic amine polymer is calculated assuming it is the corresponding free base aliphatic amine polymer and that all of the nitrogen atoms in the aliphatic amine polymer are free and not bound to any counter ions.

Alternatively, the pharmaceutically acceptable magnesium compound is present in the pharmaceutical compositions of the invention in an amount such that the molar ratio of the magnesium ion of the pharmaceutically acceptable magnesium compound to the total amine nitrogen atoms (protonated and unprotonated) of the aliphatic amine polymer is 0.4-3.0, such as 0.4-2.5, 0.8-2.0, 0.8-1.5 and 0.8-1.3. Preferably, the molar ratio is 1. This ratio is the quotient of moles of magnesium ion of the pharmaceutically acceptable magnesium compound to moles of nitrogen atom in the aliphatic amine polymer. If present, nitrogen from a counter ion or cross-linker is included in the moles of the aliphatic amine polymer.

Alternatively, the pharmaceutically acceptable magnesium compound is present in the pharmaceutical compositions of the invention in an amount such that the weight ratio of the magnesium ion of the pharmaceutically acceptable magnesium compound to the total nitrogen atom of the aliphatic amine polymer is 0.7-2.5, such as 0.7-2.0, 1.0-2.0 and 1.2-1.8. Preferably, the weight ratio is 1.57. This weight ratio is the quotient of grams of magnesium ion to grams of nitrogen atom in the aliphatic amine polymer (but not the entire composition). Thus, nitrogen from a counter ion or cross-linker, if present, is included in the grams of the nitrogen atoms in the aliphatic amine polymer.

Alternatively, the pharmaceutically acceptable magnesium compound is present in the pharmaceutical compositions of the invention in an amount such that the weight ratio of the magnesium ion of the pharmaceutically acceptable magnesium compound to the free base of the aliphatic amine polymer is 0.2-1.2, such as 0.2-1.0, 0.3-1.0, 0.3-0.8 and 0.3-0.5. Preferably, the weight ratio is 0.42. The term “the free base of the aliphatic amine polymer” is as described above. Thus, this ratio is the quotient of grams of magnesium ion to grams of aliphatic amine polymer not including any weight from any counter ion in the aliphatic amine polymer.

Aliphatic amine polymers are characterized by a repeat unit that includes at least one aliphatic amine group. Aliphatic amine groups can be part of the amine polymer backbone (e.g., a polyalkyleneimine such as polyethyleneimine) or pendant from the polymer backbone (e.g., polyallylamine), or comprise a portion of a group pendant from the polymer backbone (e.g., see Structural Formulas (7) and (8) below). Alternatively, both types of amine groups can exist within the same repeat unit and/or polymer. The word “amine,” as used herein, includes primary, secondary and tertiary amines, as well as ammonium groups such as trialkylammonium.

An aliphatic amine polymer may be obtained by polymerizing an aliphatic amine monomer. An aliphatic amine is saturated or unsaturated, straight-chained, branched or cyclic non-aromatic hydrocarbon having an amino substituent and optionally one or more additional substituents. An aliphatic amine monomer is an aliphatic amine comprising a polymerizable group such as an olefin. Suitable aliphatic amine polymers are described in U.S. Pat. Nos. 5,487,888, 5,496,545, 5,607,669, 5,618,530, 5,624,963, 5,667,775, 5,679,717, 5,703,188, 5,702,696, 5,693,675, 5,900,475, 5,925,379, 6,083,497, 6,177,478, 6,083,495, 6,203,785, 6,423,754, 6,509,013, 6,605,270, 6,726,905, 6,733,780 and 6,858,203 and U.S. Published Applications Nos. 2002/0159968 A1 and 2003/0086898 A1, the contents of which are incorporated herein by reference in their entireties.

An aliphatic amine polymer may be a homopolymer or a copolymer of one or more aliphatic amine-containing monomers or a copolymer of one or more aliphatic amine-containing monomers in combination with one or more non-amine containing monomers, which are preferably inert and non-toxic. Examples of suitable non-amine-containing monomers include vinyl alcohol, acrylic acid, acrylamide, and vinylformamide. Alternatively, an aliphatic amine polymer can be a co-polymer of two or more different aliphatic amine monomers.

Examples of aliphatic amine polymers include polymers that have one or more repeat units selected from Formulas (1)-(8):

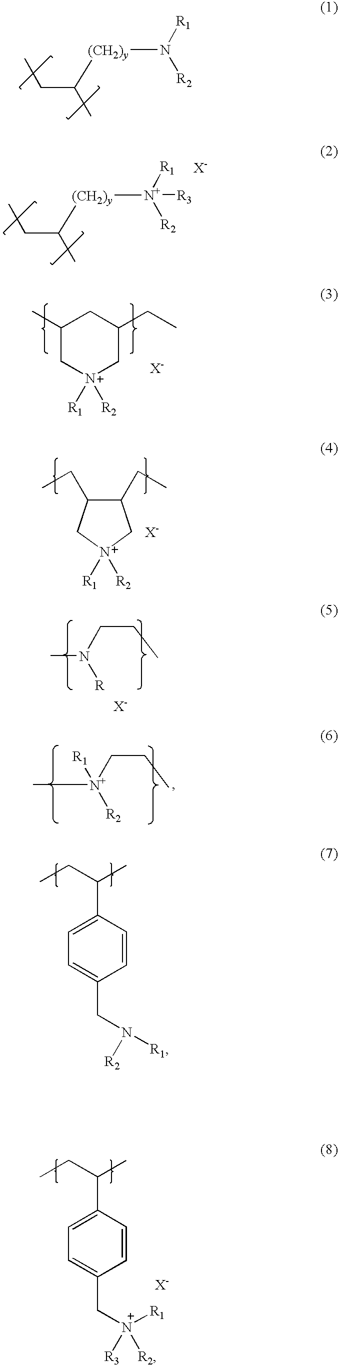

or a salt or copolymer thereof, where y is zero or an integer of one or more (e.g., between about one and about 10, preferably between one and four, more preferably one) and each R, R₁, R₂, and R₃, independently, is H, a substituted or unsubstituted alkyl group (e.g., having between 1 and 25 or between 1 and 5 carbon atoms, inclusive) or aryl (e.g., phenyl) group, and each X⁻ is an exchangeable negatively charged counterion.

Preferably, at least one of R, R₁, R₂, or R₃ is a hydrogen atom. More preferably, each of these groups is hydrogen.

The alkyl or aryl group, represented by R, R₁, R₂, and R₃, can carry one or more substituents. Suitable substituents include cationic groups, e.g., quaternary ammonium groups, or amine groups, e.g., primary, secondary or tertiary alkyl or aryl amines. Examples of other suitable substituents include hydroxy, alkoxy, carboxamide, sulfonamide, halogen, alkyl, aryl, hydrazine, guanidine, urea, poly(alkyleneimine) such as poly(ethylenimine), and carboxylic acid esters.

Preferably, an aliphatic amine polymer is a homopolymer, such as a homopolyallylamine, homopolyvinylamine, homopolydiallylamine or polyethyleneamine, but can also be a co-polymer.

In one embodiment, the aliphatic amine polymer is a homopolymer or copolymer characterized by one or more repeat units of Structural Formula (9):

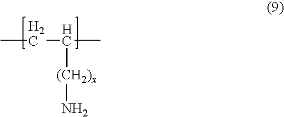

or a pharmaceutically acceptable salt thereof, where x is 0 or an integer between 1 and 4, preferably 1. The polymer represented by Structural Formula (9) is advantageously crosslinked by means of a crosslinking agent.

A preferred aliphatic amine polymer for use in the invention is polyallylamine, which is a polymer having repeat units from polymerized allyl amine monomers. The amine group of an allyl monomer can be unsubstituted or substituted with, for example, one or two C1-C10 straight chain or branched alkyl groups. These alkyl groups are optionally substituted with one or more hydroxyl, amine, halo, phenyl, amide or nitrile groups. Preferably, the aliphatic amine polymers of present invention are polyallylamine polymers comprising repeat units represented by Structural Formula (10):

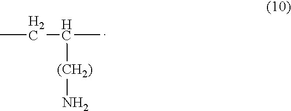

Polyallylamines that may be used as the aliphatic amine polymers of the present invention may include copolymers comprising repeat units from two or more different polymerized allyl monomers or with repeat units from one or more polymerized allyl monomers and repeat units from one or more polymerized non-allyl monomers. Examples of suitable non-allyl monomers include acrylamide monomers, acrylate monomers, maleic acid, malimide monomers, vinyl acylate monomers and alkyl substituted olefines. Alternatively, other olefinic aliphatic amine monomers can be polymerized with an alkylamine monomer. Preferably, however, the polyallylamines used in the present invention comprise repeat units solely from polymerized allyl amine monomers. More preferably, the polyallylamine polymers used in the present invention are homopolymers. Even more preferably, the polyallylamine polymers used in the present invention are homopolymers of repeat units represented by Structural Formula (10). Polyallylamine polymers used in the disclosed invention are preferably crosslinked polymers, more preferably crosslinked homopolymers.

In other embodiments, the aliphatic amine polymer can be a homopolymer or copolymer of polybutenylamine, polylysine, or polyarginine.

Preferably, the aliphatic amine polymer is rendered water-insoluble by crosslinking such as with a crosslinking agent. Suitable crosslinking agents include those with functional groups which react with the amino group of the aliphatic amine monomer. Alternatively, the crosslinking agent may contain two or more vinyl groups which undergo free radical polymerization with the aliphatic amine monomer. In some cases the aliphatic amine polymers are crosslinked after polymerization.

Aliphatic amine polymers are typically crosslinked with difunctional crosslinking agents. Examples of suitable crosslinking agents include diacrylates and dimethylacrylates (e.g., ethylene glycol diacrylate, propylene glycol diacrylate, butylene glycol diacrylate, ethylene glycol dimethacrylate, propylene glycol dimethacrylate, butylene glycol dimethacrylate, polyethyleneglycol dimethacrylate and polyethyleneglycol diacrylate), methylene bisacrylamide, methylene bismethacrylamide, ethylene bisacrylamide, ethylene bismethacrylamide, ethylidene bisacrylamide, divinylbenzene, bisphenol A, the diglycidal ether of bisphenol A, pyromellitic dianhydride, toluene diisocyanate, ethylene diamine and dimethyl succinate, dimethacrylate, and bisphenol A diacrylate. Examples of preferred difunctional crosslinking agents include epichlorohydrin, 1,4 butanedioldiglycidyl ether, 1,2 ethanedioldiglycidyl ether, 1,3-dichloropropane, 1,2-dichloroethane, 1,3-dibromopropane, 1,2-dibromoethane, succinyl dichloride, dimethylsuccinate, toluene diisocyanate, acryloyl chloride, and pyromellitic dianhydride. Epichlorohydrin is a most preferred crosslinking agent, because of its high availability and low cost. Epichlorohydrin is also advantageous because of its low molecular weight and hydrophilic nature, increasing the water-swellability and gel properties of the polyamine. Epichlorohydrin forms 2-hydroxypropyl crosslinking groups.

Other methods of inducing crosslinking on already polymerized materials include, but are not limited to, exposure to ionizing radiation, ultraviolet radiation, electron beams, radicals, and pyrolysis.

The level of crosslinking renders the aliphatic amine polymers insoluble and substantially resistant to absorption and degradation, thereby limiting the activity of the aliphatic amine polymer to the gastrointestinal tract, and reducing potential side-effects in the patient. Typically, the crosslinking agent is present in an amount 0.5-35% (such as 0.5-25%, 2.5-20% or 1-10%) by weight, based upon total weight of aliphatic amine monomer plus crosslinking agent.

Typically, between about 3% and about 30% of the allylic nitrogen atoms are bonded to a crosslinking group, preferably between 6% and about 21%.

The aliphatic amine polymers can also be further derivatized; examples include alkylated amine polymers, as described, for example, in U.S. Pat. Nos. 5,679,717, 5,607,669 and 5,618,530, the teachings of which are incorporated herein by reference in their entireties. Preferred alkylating agents include hydrophobic groups (such as aliphatic hydrophobic groups) and/or quaternary ammonium- or amine-substituted alkyl groups.

Non-crosslinked and crosslinked polyallylamine and polyvinylamine are generally known in the art and are commercially available. Methods for the manufacture of polyallylamine and polyvinylamine, and crosslinked derivatives thereof, are described in the above U.S. patents. Patents by Harada et al., (U.S. Pat. Nos. 4,605,701 and 4,528,347), which are incorporated herein by reference in their entireties, also describe methods of manufacturing polyallylamine and crosslinked polyallylamine. A patent by Stutts et al., (U.S. Pat. No. 6,180,754) describes an additional method of manufacturing crosslinked polyallylamine.

The molecular weight of aliphatic amine polymers is not believed to be critical, provided that the molecular weight is large enough so that the aliphatic amine polymer is substantially non-absorbed by the gastrointestinal tract. Typically, the molecular weight of aliphatic amine polymers is at least 1000. For example the molecular weight can be from: about 1000 to about 5 million, about 1000 to about 3 million, about 1000 to about 2 million or about 1000 to about 1 million.

The aliphatic amine polymers used in the invention may be optionally protonated, and in one embodiment, include polymers in which less than 40%, for example, less than 30%, such as less than 20% or less than 10% of the amine groups are protonated. In another embodiment 35% to 45% of the amines are protonated (e.g., approximately 40%). An example of a suitably protonated aliphatic amine polymer is sevelamer hydrochloride.

As described above, the aliphatic amine polymer can be administered in the form of a pharmaceutically acceptable salt. The term “pharmaceutically acceptable salt” refers to a salt of the aliphatic amine polymer to be administered which is prepared from pharmaceutically acceptable non-toxic acids including inorganic acids, organic acids, solvates, hydrates, or clathrates thereof. Thus, the nitrogen group in the repeat unit of the aliphatic amine polymer is protonated to create a positively charged nitrogen atom associated with a negatively charged counterion.

Examples of suitable counterions include organic ions, inorganic ions, or a combination thereof. For instance, suitable counterions include halides (e.g., F⁻, Cl⁻, Br⁻ and I⁻), CH₃OSO₃⁻, HSO₄⁻, SO₄²⁻, HCO₃⁻, CO₃²⁻, acetate, lactate, succinate, propionate, oxalate, butyrate, ascorbate, citrate, dihydrogen citrate, tartrate, taurocholate, glycocholate, cholate, hydrogen citrate, maleate, benzoate, folate, an amino acid derivative, a nucleotide, a lipid, or a phospholipid. Preferred anions are Cl⁻, HCO₃⁻, CO₃²⁻, and a combination thereof (e.g., a mixed carbonate and bicarbonate salt, a mixed carbonate and chloride salt, or a mixed bicarbonate and chloride salt). The counterions can be the same as, or different from, each other. For example, the polymer can contain two or more different types of counterions.

In a preferred embodiment, the aliphatic amine polymer used in the present invention is an epichlorohydrin crosslinked polyallylamine, such as sevelamer and colesevelam (see, for example, U.S. Pat. Nos. 6,423,754; 5,607,669; and 5,679,717, the contents of which are incorporated herein by reference). In a preferred embodiment, the polyallylamine polymer is crosslinked with epichlorohydrin and between about 9% to about 30% (preferably about 15% to about 21%) of the allylic nitrogen atoms are bonded to a crosslinking group and the anion is chloride, carbonate or bicarbonate or a mixed salt thereof.

A particularly preferred aliphatic mine polymer is polyallylamine hydrochloride crosslinked with about 9.0-9.8% w/w epichlorohydrin, preferably 9.3-9.5%, and is the active chemical component of the drug known as sevelamer HCl, sold under the tradename RENAGEL®. The structure is represented below:

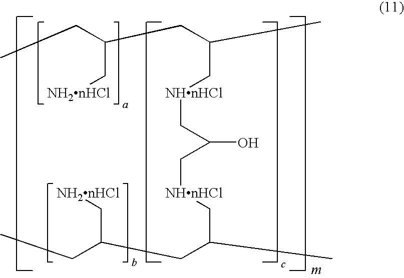

where:

the sum of a and b (the number of primary amine groups) is 9;

c (the number of crosslinking groups) is 1;

n (the fraction of protonated amines) is 0.4; and

m is a large number (to indicate extended polymer network).

Another particularly preferred aliphatic amine polymer is polyallylamine hydrochloride crosslinked with epichlorohydrin and alkylated with 1-bromodecane and (6-bromohexyl)-trimethylammonium bromide, referred to as colesevelam HCl, and marketed in the United States as WELCHOL®.

In yet another particularly preferred embodiment, the aliphatic amine polymer is a carbonate salt of sevelamer; a bicarbonate salt of sevelamer; a mixed carbonate and bicarbonate salt of sevelamer; or a mixed carbonate and chloride salt of sevelamer.

In other embodiments, a monovalent anionic source is mixed with a carbonate salt of the aliphatic amine polymer. Various examples of carbonate salts of the aliphatic amine polymer and monovalent anionic sources are disclosed in U.S. Provisional Application No. 60/624,001 “Aliphatic Amine Polymer Salts For Tableting” filed Nov. 1, 2004 and U.S. Provisional Application No. 60/628,752 “Aliphatic Amine Polymer Salts For Tableting” filed Nov. 17, 2004, the entire contents of which are incorporated herein by reference. In a preferred embodiment, the monovalent anion source is not a magnesium compound.

The monovalent anion comprises at least 0.01%, preferably 0.05%, more preferably a range of 0.01% to 2%, 0.05% to 1%, 0.08% to 0.5%, or 0.1% to 0.3% by weight of the combined weights of the carbonate salt of aliphatic amine polymer and the monovalent anion source.

Examples of suitable monovalent anions include organic ions, inorganic ions, or a combination thereof, such as halides (Cl⁻, I⁻, F⁻ and Br⁻), CH₃OSO₃⁻, HSO₄⁻, acetate, lactate, butyrate, propionate, sulphate, citrate, tartrate, nitrate, sulfonate, oxalate, succinate or palmoate. Preferred monovalent anions are halides, most preferably chloride.

Also, the monovalent anion source can be a pharmaceutically acceptable acid, ammonium or metal salt of a monovalent anion. Preferably, the metal salt is not a magnesium salt. Preferred examples of the monovalent anion source include sodium chloride and hydrochloric acid. In one preferred embodiment, the formulations of the invention comprise a carbonate salt of sevelamer and sodium chloride. In another preferred embodiment, the formulations of the invention comprise a carbonate salt of sevelamer and hydrochloric acid.

In yet another embodiment, when a carbonate salt of an aliphatic amine polymer is included in the pharmaceutical compositions of the invention, the monovalent anion source can be a monovalent anion salt of an aliphatic amine polymer comprising a repeat unit represented by Structural Formulas (1)-(10) above. In this embodiment, a monovalent anion salt of an aliphatic amine polymer and the carbonate salt of an aliphatic amine polymer can be physically mixed together. Alternatively, a single aliphatic amine polymer can comprise both carbonate and monovalent anions to form a mixed carbonate and monovalent anion salt of the single aliphatic amine polymer. When a monovalent anion salt of an aliphatic amine polymer and a carbonate salt of an aliphatic amine polymer are physically mixed together, the monovalent anion salt of an aliphatic amine polymer can be the same or a different aliphatic amine polymer as the aliphatic amine polymer carbonate salt.

As used herein, the phrase “the pharmaceutically acceptable magnesium compound entrained within the crosslinked aliphatic amine polymer” means that the crosslinked aliphatic amine polymer encaptures the pharmaceutically acceptable magnesium compound, for example, within a pocket (or pockets) generated by crosslinking. A crosslinked aliphatic amine polymer entrained with a pharmaceutically acceptable magnesium compound can be prepared by crosslinking an aliphatic amine polymer as described above in the presence of a pharmaceutically acceptable magnesium compound. For example, a polyallylamine can be crosslinked by multifunctional crosslinking agent(s), such as epichlorohydrin, in the presence of magnesium oxide to form a crosslinked polyallylamine entrained with magnesium oxide. Various examples and preferred values for the aliphatic amine polymers, crosslinking agents and pharmaceutically acceptable magnesium compounds are as described above. Typically, when a crosslinked aliphatic amine polymer entrained with a pharmaceutically acceptable magnesium compound is employed, the crosslinking agent is present in an amount 0.5-35% (such as 0.5-30%, 2.5-30%, 5-25%, 5-20% or 5-15%) by weight, based upon total weight of aliphatic amine monomer plus crosslinking agent.

The pharmaceutical compositions of the invention optionally include one or more pharmaceutically acceptable carriers and/or diluents therefor, such as lactose, starch, cellulose and dextrose. Other excipients, such as flavoring agents; sweeteners; and preservatives, such as methyl, ethyl, propyl and butyl parabens, can also be included.

The carriers, diluents and/or excipients are “acceptable” in the sense of being compatible with the other ingredients of the pharmaceutical composition and not deleterious to the recipient thereof. The pharmaceutical compositions can conveniently be presented in unit dosage form and can be prepared by any suitable method known to the skilled artisan. In general, the pharmaceutical compositions are prepared by uniformly and intimately bringing into association the aliphatic amine polymer and pharmaceutically acceptable magnesium compound with the carriers, diluents and/or excipients and then, if necessary, dividing the product into unit dosages thereof.

The pharmaceutical compositions of the invention can be formulated as a tablet, sachet, slurry, food formulation, troche, capsule, elixir, suspension, syrup, wafer, chewing gum or lozenge. A syrup formulation will generally consist of a suspension or solution of the phosphate binding polymer or salt in a liquid carrier, for example, ethanol, glycerine or water, with a flavoring or coloring agent. Where the composition is in the form of a tablet, one or more pharmaceutical carriers routinely used for preparing solid formulations can be employed. Examples of such carriers include magnesium stearate, starch, lactose and sucrose. Where the composition is in the form of a capsule, the use of routine encapsulation is generally suitable, for example, using the aforementioned carriers in a hard gelatin capsule shell. Where the composition is in the form of a soft gelatin shell capsule, pharmaceutical carriers routinely used for preparing dispersions or suspensions can be considered, for example, aqueous gums, celluloses, silicates or oils, and are incorporated in a soft gelatin capsule shell.

In a preferred embodiment, the pharmaceutical compositions of the invention are formulated as a tablet.

In another preferred embodiment, the pharmaceutical compositions of the invention are formulated as a powder formulation which can be easily packaged as a sachet or a tub from which a unit dose is measured by, e.g., a spoon or cup, or an instrument capable of dispensing a pre-defined dosage amount. The powder formulation preferably further includes a pharmaceutically acceptable anionic polymer, such as alginate (e.g., sodium alginate, potassium alginate, calcium alginate, magnesium alginate, ammonium alginate, esters of alginate, etc.), carboxymethyl cellulose, poly lactic acid, poly glutamic acid, pectin, xanthan, carrageenan, furcellaran, gum arabic, karaya gum, gum ghatti, gum carob and gum tragacanth (see U.S. Provisional Application No. 60/717,200 filed on Sep. 15, 2005, the entire teachings of which are incorporated herein by reference). One or more sweeteners and/or flavorants can be optionally included in the powder formulation.

Though the above description is directed toward routes of oral administration of pharmaceutical compositions consistent with embodiments of the invention, it is understood by those skilled in the art that other modes of administration using vehicles or carriers conventionally employed and which are inert with respect to the aliphatic amine polymers and pharmaceutically acceptable magnesium compounds may be utilized for preparing and administering the pharmaceutical compositions. Illustrative of such methods, vehicles and carriers are those described, for example, in Remington's Pharmaceutical Sciences, 18^{th} ed. (1990), the disclosure of which is incorporated herein by reference.

Still other embodiments of the invention are directed to compositions comprising an aliphatic amine polymer or a salt thereof, and a pharmaceutically acceptable magnesium compound comprising a magnesium ion. Suitable examples and preferred values for the aliphatic amine polymers and pharmaceutically acceptable magnesium compounds are as described above for the pharmaceutical compoistions of the invention.

In one embodiment, the pharmaceutically acceptale magnesium ion comprises 5-35% (e.g., 10-30%, 10-25%, 13-25%, 15-22% and 16-20%), by anhydrous weight of the composition.

Alternatively, the pharmaceutically acceptale magnesium ion of the pharmaceutically acceptable magnesium compound comprises 5-35% (e.g., 10-30%, 10-25%, 13-25%, 15-22% and 16-20%), by anhydrous weight of the combined weight of the magnesium compound and the free base of the aliphatic amine polymer. Herein, the term “the free base of the aliphatic amine polymer” means the aliphatic amine polymer not including any counter ion. When the quantity of magnesium compound in the composition is expressed in this fashion, it is to be understood that the aliphatic amine polymer in the composition can be unprotonated, partially protonated or completely protonated. However, the weight of the aliphatic amine polymer is calculated assuming it is the corresponding free base aliphatic amine polymer and that all of the nitrogen atoms in the aliphatic amine polymer are free and not bound to any counter ions.

Alternatively, the pharmaceutically acceptable magnesium compound is present in the compositions of the invention in an amount such that the molar ratio of the magnesium ion of the pharmaceutically acceptable magnesium compound to the total amine nitrogen atoms (protonated and unprotonated) of the aliphatic amine polymer is 0.4-3.0, such as 0.4-2.5, 0.8-2.0, 0.8-1.5 and 0.8-1.3. Preferably, the molar ratio is 1. This ratio is the quotient of moles of magnesium ion of the pharmaceutically acceptable magnesium compound to moles of nitrogen atom in the aliphatic amine polymer. If present, nitrogen from a counter ion or cross-linker is included in the moles of the aliphatic amine polymer.

Alternatively, the pharmaceutically acceptable magnesium compound is present in the compositions of the invention in an amount such that the weight ratio of the magnesium ion of the pharmaceutically acceptable magnesium compound to the total nitrogen atom of the aliphatic amine polymer is 0.7-2.5, such as 0.7-2.0, 1.0-2.0 and 1.2-1.8. Preferably, the weight ratio is 1.57. This weight ratio is the quotient of grams of magnesium ion to grams of nitrogen atom in the aliphatic amine polymer (but not the entire composition). Thus, nitrogen from a counter ion or cross-linker, if present, is included in the grams of the nitrogen atoms in the aliphatic amine polymer.

Alternatively, the pharmaceutically acceptable magnesium compound is present in the compositions of the invention in an amount such that the weight ratio of the magnesium ion of the pharmaceutically acceptable magnesium compound to the free base of the aliphatic amine polymer is 0.2-1.2, such as 0.2-1.0, 0.3-1.0, 0.3-0.8 and 0.3-0.5. Preferably, the weight ratio is 0.42. The term “the free base of the aliphatic amine polymer” is as described above. Thus, this ratio is the quotient of grams of magnesium ion to grams of aliphatic amine polymer not including any weight from any counter ion in the aliphatic amine polymer.

In another embodiment, a composition of the invention comprises an aliphatic amine polymer or a salt thereof, and a pharmaceutically acceptable magnesium compound comprising a magnesium ion, where the magnesium compound is selected from the group consisting of magnesium oxide, magnesium hydroxide, magnesium carbonate, magnesium formate, and a combination thereof. In yet another embodiment, the present invention is directed to a composition comprising a crosslinked aliphatic amine polymer or a salt thereof, and a pharmaceutically acceptable magnesium compound comprising a magnesium ion, where the magnesium compound is entrained within the crosslinked aliphatic amine polymer.

The pharmaceutical compositions of the invention disclosed herein can be used for treating hyperphosphatemia in a subject. Hyperphosphatemia is typically defined for humans as a serum phosphate level of greater than about 4.5 mg/dL. The condition, especially if present over extended periods of time, leads to severe abnormalities in calcium and phosphorus metabolism and can be manifested by aberrant calcification in joints, lungs and eyes. Elevated serum phosphate is commonly present in patients with renal insufficiency, hypoparathyroidism, pseudohypoparathyroidism, acute untreated acromegaly, overmedication with phosphate salts, and acute tissue destruction as occurs during rhabdomyolysis and treatment of malignancies.

As used herein a subject is a mammal, preferably a human, but can also be an animal in need of veterinary treatment, such as a companion animal (e.g., dogs, cats, and the like), a farm animal (e.g., cows, sheep, pigs, horses, and the like) or a laboratory animal (e.g., rats, mice, guinea pigs, and the like). A subject “in need of treatment” includes a subject with chronic renal failure. Other examples of subjects in need of treatment include patients with a disease associated with disorders of phosphate metabolism. Examples of diseases and/or disorders of this type include hyperparathyroidism, inadequate renal function, and hyperphosphatemia.

An “effective amount” of a pharmaceutical composition disclosed above is a quantity that results in a beneficial clinical outcome of or exerts an influence on, the condition being treated with the pharmaceutical composition compared with the absence of treatment. The administering amount of a pharmaceutical composition disclosed above to the subject will depend on the degree, severity, and type of the disease or condition, the amount of therapy desired, and the release characteristics of the pharmaceutical composition. It will also depend on the subject's health, size, weight, age, sex, and tolerance to drugs. Typically, the pharmaceutical compositions of the invention are administered for a sufficient period of time to achieve the desired therapeutic effect. Typically between about 5 mg per day and about 15 g per day of a pharmaceutical composition disclosed above (alternatively between about 50 mg per day and about 12 g per day, alternatively between about 0.5 g per day and about 12 g per day, alternatively between about 1 g per day and about 12 g per day, alternatively between about 0.5 g per day and about 10 g per day, alternatively between about 1 g per day and about 10 g per day, alternatively between about 2 g per day and about 10 g, alternatively between about 3 g per day and about 10 g per day, alternatively between about 1 g per day and about 8 g per day, alternatively between about 2 g per day and about 8 g per day, alternatively between about 2 g per day and about 6 g per day, alternatively between about 2 g per day and about 5 g per day) is administered to the subject in need of treatment. These dosages can be administered several times/day (e.g., 2, 3, 4 or 5 times/day) or once/day. The pharmaceutical compositions of the invention can be administered at least four times per day with meals, at least three times per day with meals, at least twice per day with meals, at least once per day with meals, (see U.S. Provisional Application No. 60/623,985, “Once a day formulation for phosphate binders” filed Nov. 1, 2004, the entire contents of which are incorporated herein by reference). In one specific example, about 0.8-7.2 g (e.g., 1.2 g, 1.6 g, 1.8 g, 2.0, 2.4 g, 3.0 g, 3.2 g, 3.6 g, 4.0 g or 4.8 g per dose for 2-3 times per day, or 3.0 g, 3.2 g, 3.6 g, 4.0 g or 4.8 g, 5.4 g, 6.0 g, 6.2 g, 6.6 g, 7.0 g or 7.2 g per dose for once per day) of a pharmaceutical composition of the invention is administered per day.

Typically, the pharmaceutical compositions of the invention can be administered before or after a meal, or with a meal. As used herein, “before” or “after” a meal is typically within two hours, preferably within one hour, more preferably within thirty minutes, most preferably within ten minutes of commencing or finishing a meal, respectively.

In one preferred embodiment, the method of the present invention is a mono-therapy where the pharmaceutical compositions of the invention are used alone. Accordingly, in this embodiment, the aliphatic amine polymer or pharmaceutically acceptable salt thereof, and the pharmaceutically acceptable magnesium compound are the only pharmaceutically active ingredient, e.g., the only phosphate binders, in the pharmaceutical compositions. In this embodiment, calcium- and aluminum-based phosphate binders are excluded from the pharmaceutical compositions. Similarly, with respect to the disclosed methods, the aliphatic amine polymer and pharmaceutically acceptable magnesium compound are the only phosphate binders administered to a subject.

The method of the present invention also includes a co-therapy with other therapeutically active drugs, such as a phosphate transport inhibitor, HMG-CoA reductase inhibitor or an alkaline phosphatase inhibitor. A phosphate transport inhibitor, HMG-CoA reductase inhibitor or an alkaline phosphatase inhibitor, and the aliphatic amine polymer and pharmaceutically acceptable magnesium compound can be co-formulated in a single formulation, or alternatively co-administered in separate formulations.

Suitable examples of phosphate transport inhibitors can be found in co-pending U.S. Application Publication Nos. 2004/0019113 and 2004/0019020 and WO 2004/085448, the entire teachings of each of these are incorporated herein by reference.

The invention is illustrated by the following examples which are not intended to be limiting in any way.

### EXEMPLIFICATION

### Example 1

### Preparation of Admixture of MgO and Sevelamer

MgO (0.1 g) was added to sevelamer hydrochloride (1 g) and mixed. Anal. Found: C, 42.74; H, 8.69; N, 14.85; Cl, 15.77; Mg, 6.16.

### Example 2

### Preparation of Admixture of MgO and Sevelamer

MgO (0.2 g) was added to sevelamer hydrochloride (1 g) and mixed. Anal. Found: C, 39.52; H, 8.64; N, 13.74; Cl, 14.94; Mg, 14.62.

### Example 3

### Preparation of Admixture of MgO and Sevelamer

MgO (0.5 g) was added to sevelamer hydrochloride (1 g) and mixed. Anal. Found: C, 33.23; H, 7.41; N, 11.51; Cl, 11.25; Mg, 39.31.

### Example 4

### Preparation of Admixture of MgO and Sevelamer

MgO (1.0 g) was added to sevelamer hydrochloride (1 g) and mixed. Anal. Found: C, 24.83; H, 5.59; N, 8.50; Cl, 9.18; Mg, 47.90.

### Example 5

### Preparation of Admixture of MgO and Sevelamer

MgO (5.0 g) was added to sevelamer hydrochloride (1 g) and mixed. Anal. Found: C, 12.37; H, 2.90; N, 4.12; Cl, 3.03; Mg, 60.81.

### Example 6

### Preparation of Admixture of Epichlorohydrin-Crosslinked Polyallylamine and MgO

### A. Preparation of the 10% Epichlorohydrin-Crosslinked Polyallylamine: 271.2 g PAA.HCl, 10.0 mol % Epichlorohydrin (on the Basis of the Molecular Weight of a Repeat Unit of Polyallylamine)

To a solution of polyallylamine hydrochloride (PAA.HCl, 50% (w/w) aqueous solution) was added deionized water (1050 g) followed by NaOH (185.38 g of 50% (w/w) NaOH in water) to form a partially neutralized polyallylamine solution. This solution contains the equivalent of 18.08% (w/w) polyallylamine hydrochloride.

To a partially neutralized polyallylamine hydrochloride (PAA.HCl) solution (1500 g) was added epichlorohydrin (22.8 mL). A gel was formed within 30 minutes. After curing at room temperature overnight the gel was broken into small pieces and placed onto a large plastic Buchner funnel with filter paper. Under vacuum, the polymer gel was washed 12 times (4 L each wash). The washed polymer was dried in a forced-air oven at 60° C. to afford 247.54 g. The dried polymer was ground in a Fritsch grinder using a number 2 blade and sieved through an 80 mesh sieve to afford 178.64 g of −80 mesh material (Sample A) and 18.17 g of +80 mesh material (Sample B). Anal. Found for Sample A: C, 42.76; H, 10.12; N, 14.58; Cl, 18.28.

### B. Preparation of Admixture of Epichlorohydrin-Crosslinked Polyallylamine and MgO

A sample of 10% epichlorohydrin crosslinked polyallylamine (5.4 g of Sample A) was intimately mixed with MgO (2.1 g, −325 mesh).

### Example 7

### Preparation of Polyallylamine Crosslinked in the Presence of Magnesium Compound: 50 g PAA.HCl, 70.5 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (35.25 g). After stirring for 1 hour at room temperature, epichlorohydrin (4.10 mL) was added. A gel was formed within 30 minutes. After curing at room temperature over 3 nights the gel was broken into small pieces and suspended into deionized water (4 L). After stirring for 20 minutes, the suspension was filtered. The filtered polymer was washed two times with deionized water (4 L each wash). Half of the filtered polymer was dried in a forced-air oven at 60° C. to afford 37.37 g (Example 7-#1). Anal. Found for Example 7-#1: C, 25.31; H, 7.09; N, 8.37; Cl, 3.44; Mg, 26.76. The other half of the filtered polymer was lyophilized to afford 36.57 g Example 7-#2). Anal. Found for Example 7-#2: C, 29.71; H, 8.09; N, 9.90; Cl, 3.29; Mg, 5.23.

### Example 8

### Preparation of Polyallylamine Crosslinked in the Presence of Magnesium Compound: 50 g PAA.HCl 53 wt % MgO (on the Basis of the Weight of PAA.HCl), 9.8 mol % Epichlorohydrin (on the Basis of the Molecular Weight of a Repeat Unit of Polyallylamine)

This sample was prepared as described above in Example 7, except the amount of MgO used. To a partially neutralized polyallylamine hydrochloride solution (see Example 6, 276.5 g) was added MgO (26.44 g). After stirring for 1 hour at room temperature, epichlorohydrin (4.10 mL) was added. A gel was formed within 30 minutes. After curing at room temperature over 3 nights the gel was broken into small pieces and suspended into deionized water (4 L). After stirring for 20 minutes, the suspension was filtered. The filtered polymer was washed two times with deionized water (4 L each wash). Half of the filtered polymer was dried in a forced-air oven at 60° C. to afford 32.91 g (Example 8-#1). Anal. Found for Example 8-#1: C, 32.26; H, 8.40; N, 10.85; Cl, 4.46; Mg, 16.84. The other half of the filtered polymer was lyophilized to afford 31.18 g (Example 8-#2). Anal. Found for Example 8-#2: C, 27.36; H, 7.76; N, 9.28; Cl, 3.65; Mg, 12.74.

### Example 9

### Preparation of Polyallylamine Crosslinked in the Presence of Magnesium Compound: 100 g PAA.HCl, 53 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 553 g) was added MgO (52.88 g). After stirring for 1 h at room temperature epichlorohydrin (8.2 mL) was added. A gel was formed within 30 min. After curing at room temperature the gel was broken into small pieces and suspended into deionized water (4 L). After stirring for 20 minutes the suspension was filtered. The filtered polymer was washed three more times with deionized water (4 L each wash). The filtered polymer was dried in a forced-air oven at 60° C. to afford 135.45 g. The polymer was ground in a coffee mill and sieved using an 80 mesh sieve to afford 61.82 g of +80 mesh material (Example 9-#2) and 73.58 g of −80 mesh material (Example 9-#1). The polymer Example 9-#2 was further ground in a Fritsch grinder using a # 2 screen and sieved using an 80 mesh sieve to afford 32.81 g of +80 mesh material (Example 9-#4) and 28.26 g of −80 mesh material (Example 9-#3). Anal. Found: Example 9-#1, C, 28.58; H, 7.69; N, 9.54; Cl, 3.60; Mg, 19.37.

### Example 10

### Preparation of Polyallylamine Crosslinked in the Presence of Magnesium Compound: 200 g PAA.HCl, 53 wt % MgO (on the Basis of the Weight of PAA.HCl), 9.8 mol % Epichlorohydrin (on the Basis of the Molecular Weight of a Repeat Unit of Polyallylamine)

This sample was prepared as described above in Example 9, but in a larger scale. Anal. Found: C, 28.55; H, 7.99; N, 9.72; Cl, 6.44; Mg, 18.97.

### Example 11

### Preparation of Polyallylamine Crosslinked in the Presence of Magnesium Compound: 50 g PAA.HCl, 53 wt % MgO (30 mesh) (on the Basis of the Weight of PAA.HCl), 9.8 mol % Epichlorohydrin (on the Basis of the Molecular Weight of a Repeat Unit of Polyallylamine)

This preparation was performed in a similar manner as described in Example 8. To a partially neutralized polyallylamine solution (585 g) was added MgO (36.85 g, beads in about 30 mesh). After stirring for 1 minute at room temperature, epichlorohydrin (5.71 mL) was added. A gel was formed. After curing at room temperature over 1 hour, the gel was broken into small particles, washed with deionized water (4×4 L), and dried in a forced-air oven at 60° C. to afford 92.93 g.

### Example 12

### Preparation of Polyallylamine Crosslinked in the Presence of Magnesium Compound: 50 g PAA.HCl, 157 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (79.32 g, −325 mesh). After stirring for 1 hour at room temperature, epichlorohydrin (4.10 mL) was added. A gel was formed. After curing at room temperature overnight, the gel was broken into small pieces, washed with deionized water (3×4 L), and dried in a forced-air oven at 60° C. to afford 134.58 g.

### Example 13

### Preparation of Polyallylamine Crosslinked in the Presence of Magnesium Compound: 50 g PAA.HCl, 211 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (105.76 g, −325 mesh). After stirring for 1 hour at room temperature, epichlorohydrin (4.10 mL) was added. A gel was formed. After curing at room temperature overnight, the gel was broken into small pieces, washed with deionized water (3×4 L), and dried in a forced-air oven at 60° C. to afford 170.27 g.

### Example 14

### Preparation of Polyallylamine Crosslinked in the Presence of Magnesium Compound: 24 g PAA, 20 wt % MgO (on the Basis of the Weight of PAA.HCl), 9.8 Mol % Epichlorohydrin (on the Basis of the Molecular Weight of a Repeat Unit of Polyallylamine)

To a partially neutralized solution of polyallylamine hydrochloride (see Example 6, 200 g, 50 wt. % aqueous solution) in deionized water (200 g) was added 50% aqueous NaOH until the solution had pH 13. This solution was dialyzed (MWCO 6-8000) against deionized water, and lyophilized to afford 53.86 g of polyallylamine free base.

To a mixture of polyallylamine free base (23.54 g, 629-017), deionized water (94.16 g), and MgO (4.69 g, −325 mesh) was added epichlorohydrin (3.16 mL). A gel formed after 20 minutes, and was allowed to cure at room temperature overnight. The gel was broken into small pieces and dried in a forced-air oven at 60° C. to afford 32.79 g.

### Example 15

### Preparation of Polyallylamine Crosslinked in the Presence of Magnesium Compound: 10 g PAA.HCl, 71 wt % MgO (on the Basis of the Weight of PAA.HCl) 9.8 mol % Epichlorohydrin (on the Basis of the Molecular Weight of a Repeat Unit of Polyallylamine)

To a partially neutralized polyallylamine hydrochloride solution (see Example 6, 55.3 g) was added MgO (7.05 g, −325 mesh). After stirring for 1 hour at room temperature, epichlorohydrin (0.97 g) was added. A gel was formed. After curing at room temperature, the gel was broken into small pieces, washed with deionized water (3×1 L), and lyophilized to afford 10.5 g. Anal. Found: C, 27.25; H, 7.05; N, 9.15; Mg, 23.40.

### Example 16

### Preparation of Polyallylamine Crosslinked in the Presence of Magnesium Compound: 10 g PAA.HCl, 35 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 55.3 g) was added MgO (3.53 g, −325 mesh). After stirring for 1 hour at room temperature, epichlorohydrin (0.97 g) was added. A gel was formed. After curing at room temperature, the gel was broken into small pieces, washed with deionized water (3×1 L), and lyophilized to afford 9.45 g. Anal. Found: C, 43.35; H, 9.34; N, 14.72; Mg, 13.23.

### Example 17

### Preparation of Polyallylamine Crosslinked in the Presence of Magnesium Compound: 50 g PAA.HCl, 18 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 275.86 g) was added MgO (8.8 g, −325 mesh). After stirring for 2 hours at room temperature, epichlorohydrin (4.86 g) was added. A gel was formed. After curing at room temperature, the gel was broken into small pieces, washed with deionized water (6×2 L), and lyophilized to afford 39.95 g. Anal. Found: C, 51.68; H, 11.45; N, 17.86; Cl, 4.83; Mg, 5.9.

### Example 18

### Preparation of Polyallylamine Crosslinked in the Presence of Magnesium Compound: 10 g PAA.HCl, 7 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 55.3 g) was added MgO (0.7 g, −325 mesh). After stirring for 1 hour at room temperature, epichlorohydrin (0.97 g) was added. A gel was formed. After curing at room temperature, the gel was broken into small pieces, washed with deionized water (3×1 L), and lyophilized to afford 7.95 g. Anal. Found: C, 49.37; H, 9.98; N, 16.70; Mg, 0.61.

### Example 19

### Preparation of Polyallylamine Crosslinked in the Presence of Magnesium Compound: 10 g PAA.HCl, 3.5 wt % MgO (on the Basis of the Weight of PAA.HCl), 9.8 mol % Epichlorohydrin (on the Basis of the Molecular Weight of a Repeat Unit of Polyallylamine)

This sample was prepared as described above except for using 0.35 g of MgO (−325 mesh). After stirring for 1 hour at room temperature, epichlorohydrin (0.97 g) was added. 8.25 g of a lyophilized gel was obtained. Anal. Found: C, 47.05; H, 10.00; N, 16.06; Mg, 0.29.

### Example 20

### Preparation of Polyallylamine Crosslinked in the Presence of Magnesium Compound: 100 g PAA.HCl, 53 wt % MgO heavy (on the Basis of the Weight of PAA.HCl), 9.8 mol % Epichlorohydrin (on the Basis of the Molecular Weight of a Repeat Unit of Polyallylamine)

This sample was prepared in a similar manner as described in Example 8, except for using MgO heavy. To partially neutralized polyallylamine hydrochloride solution (see Example 6, 553 g) was added MgO, heavy (52.88 g). After stirring for 1 hour at room temperature, epichlorohydrin (8.2 mL) was added. A gel was formed within 30 minutes. After curing at room temperature the gel was broken into small pieces and suspended into deionized water (4 L). After stirring for 20 minutes, the suspension was filtered. The filtered polymer was washed with deionized water (3×4 L). The filtered polymer was dried in a forced-air oven at 60° C. to afford 150.2 g. The polymer was ground in a coffee mill and sieved using an 80 mesh sieve to afford 118.56 g of +80 mesh material (Example 20-# 1) and 32.06 g of −80 mesh material Example 20-#2). Anal. Found: Example 20-#2, C, 27.91; H, 7.60; N, 9.35; Cl, 8.52; Mg, 18.23. Anal. Found: Example 20-#1, 27.33; H, 7.50; N, 9.35; Cl, 7.86; Mg, 19.89.

### Example 21

### Preparation of Polyallylamine Crosslinked in the Presence of Magnesium Compound: 100 g PAA.HCl, 53 wt % MgO light (on the Basis of the Weight of PAA.HCl), 9.8 mol % Epichlorohydrin (on the Basis of the Molecular Weight of a Repeat Unit of Polyallylamine)

This sample was prepared as described above in Example 27 except for using MgO light instead of MgO heavy). 154.6 g of dried polymer gel was obtained. The polymer was ground in a coffee mill and sieved using an 80 mesh sieve to afford 122.96 g of +80 mesh material (Example 21-#1) and 31.64 g of −80 mesh material (Example 21-#2). Anal. Found: Example 21-#2, C, 27.40; H, 7.50; N, 9.19; Cl, 7.76; Mg, 18.82. Anal. Found: Example 21-#1, C, 27.30; H, 7.63; N, 9.34; Cl, 8.86; Mg, 18.80

### Example 22

### Preparation of Polyallylamine Crosslinked in the Presence of Magnesium Compound: 50 g PAA.HCl, 35.3 wt % MgO (on the Basis of the Weight of PAA.HCl), 14.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (17.65 g). After stirring for 1 hour at room temperature, epichlorohydrin (6.15 mL) was added. A gel was formed within 20 minutes. After curing at room temperature over 3 nights the gel was broken into small pieces and suspended into deionized water (4 L). After stirring for 20 minutes, the suspension was filtered. The filtered polymer was washed with deionized water (2×4 L). Half of the filtered polymer was dried in a forced-air oven at 60° C. to afford 26.88 g (Example 22#1). Anal. Found: Example 22-#1, C, 35.10; H, 8.30; N, 11.33; Cl, 3.69; Mg, 24.82. The other half of the filtered polymer was lyophilized to afford 26.35 g (Example 22-#2). Anal. Found: Example 22-#2, C, 33.97; H, 8.44; N, 11.14; Cl, 3.42; Mg, 23.08.

### Example 23

### Preparation of Polyallylamine Crosslinked in the Presence of Magnesium Compound: 50 g PAA.HCl 35.3 wt % MgO (on the Basis of the Weight of PAA.HCl), 19.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (17.65 g). After stirring for 1 hour at room temperature, epichlorohydrin (8.20 mL) was added. A gel was formed within 20 minutes. After curing at room temperature over 3 nights the gel was broken into small pieces and suspended into deionized water (4 L). After stirring for 20 minutes, the suspension was filtered. The filtered polymer was washed with deionized water (2×4 L). Half of the filtered polymer was dried in a forced-air oven at 60° C. to afford 28.09 g (Example 23-#1). Anal. Found: Example 23-#1, C, 24.60; H, 6.87; N, 7.50; Cl, 5.16; Mg, 8.67. The other half of the filtered polymer was lyophilized to afford 26.97 g (Example 2342). Anal. Found: Example 23-#2, C, 41.79; H, 9.54; N, 13.26; Cl, 3.80; Mg, 24.00

### Example 24

### Preparation of Polyallylamine Crosslinked in the Presence of Magnesium Compound: 200 g PAA.HCl, 53 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 1106 g) was added MgO (105.76 g). After stirring for 1 hour at room temperature, epichlorohydrin (25.10 mL) was added. A gel was formed within 10 minutes. After curing at room temperature over 3 nights, the gel was broken into small particles, washed with deionized water, and dried in a forced-air oven at 60° C. to afford 278.13 g of product.

### Example 25

### Preparation of Polyallylamine Crosslinked in the Presence of Magnesium Compound: 200 g PAA.HCl, 53 wt % MgO (on the Basis of the Weight of PAA.HCl), 20 mol % Epichlorohydrin (on the Basis of the Molecular Weight of a Repeat Unit of Polyallylamine)

To a partially neutralized polyallylamine hydrochloride solution (see Example 6, 1106 g) was added MgO (105.76 g). After stirring for 1 hour at room temperature, epichlorohydrin (33.47 mL) was added. A gel was formed. After curing at room temperature over 3 nights, the gel was broken into small particles, washed with deionized water, and dried in a forced-air oven at 60° C. to afford 267.52 g of product.

### Example 26

### Preparation of Polyallylamine Crosslinked in the Presence of Magnesium Compound: 200 g PAA.HCl, 53 wt % MgO (on the Basis of the Weight of PAA.HCl), 30 mol % Epichlorohydrin (on the Basis of the Molecular Weight of a Repeat Unit of Polyallylamine)

To a partially neutralized polyallylamine hydrochloride solution (see Example 6, 1106 g) was added MgO (105.76 g). After stirring for 1 hour at room temperature, epichlorohydrin (50.21 mL) was added. A gel was formed. After curing at room temperature over 3 nights, the gel was broken into small particles, washed with deionized water, and dried in a forced-air oven at 60° C. to afford 295.19 g of product.

### Example 27

### Preparation of Polyallylamine Crosslinked in the Presence of Magnesium Compound: 200 g PAA.HCl, 53 wt % MgO (on the Basis of the Weight of PAA.HCl), 5 mol % Epichlorohydrin (on the Basis of the Molecular Weight of a Repeat Unit of Polyallylamine)

To a partially neutralized polyallylamine hydrochloride solution (see Example 6, 1106 g) was added MgO (105.76 g). After stirring for 1 hour at room temperature, epichlorohydrin (8.37 mL) was added. A gel was formed. After curing at room temperature over 3 nights, the gel was broken into small particles, washed with deionized water, and dried in a forced-air oven at 60° C. to afford 268.41 g of product

### Example 28

### Preparation of Polyallylamine Crosslinked in the Presence of Magnesium Compound: 200 g-PAA.HCl, 53 wt % MgO (on the Basis of the Weight of PAA.HCl), 50 mol % Epichlorohydrin (on the Basis of the Molecular Weight of a Repeat Unit of Polyallylamine)

To a partially neutralized polyallylamine hydrochloride solution (see Example 6, 1106 g) was added MgO (105.76 g). After stirring for 1 hour at room temperature, epichlorohydrin (83.66 mL) was added. A gel was formed. After curing at room temperature over 3 nights, the gel was broken into small particles, washed with deionized water, and dried in a forced-air oven at 60° C. to afford 285.2 g of product.

### Example 29

### Preparation of Polyallylamine Crosslinked in the Presence of Magnesium Compound: 50 g PAA.HCl, 106 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 276.5 g) was added MgO (52.88 g, −325 mesh). After stirring for 1 hour at room temperature, epichlorohydrin (4.10 mL) was added. A gel was formed. After curing at room temperature overnight, the gel was broken into small pieces, washed with deionized water (3×4 L), and dried in a forced-air oven at 60° C. to afford 97.76 g of product.

### Example 30

### Preparation of Polyallylamine Crosslinked in the Presence of Magnesium Compound: 50 g PAA.HCl, 53 wt % MgO (on the Basis of the Weight of PAA.HCl), 1 mol % Epichlorohydrin (on the Basis of the Molecular Weight of a Repeat Unit of Polyallylamine)

To a partially neutralized polyallylamine hydrochloride solution (see Example 6, 276.5 g) was added MgO (26.44 g, −325 mesh). After stirring for 1 hour at room temperature, epichlorohydrin (0.419 mL) was added. A gel was formed. After curing at room temperature overnight, the gel was broken into small pieces, washed with deionized water, and dried in a forced-air oven at 60° C. to afford 52.28 g of product.

### Example 31

### Preparation of Polyallylamine Crosslinked in the Presence of Magnesium Compound: 50 g PAA.HCl, 50 wt % MgO (on the Basis of the Weight of PAA.HCl), 10 mol % Bis(2-chloroethyl)amine (on the Basis of the Molecular Weight of a Repeat Unit of Polyallylamine)

A mixture of a partially neutralized polyallylamine hydrochloride solution (see Example 6, 276.5 g), MgO (25 g, −325 mesh), and bis(2-chloroethyl)amine hydrochloride (9.46 g) was heated at 60° C. for 8 hours. A gel formed after 15 minutes. After cooling to room temperature, the gel was broken into small pieces, washed with deionized water (3×4 L), and dried in a forced-air oven at 60° C. to afford 60.56 g.

### Example 32

### Preparation of Polyallylamine Crosslinked in the Presence of Magnesium Compound: 50 g PAA.HCl, 100 wt % MgO (on the Basis of the Weight of PAA.HCl), 10 mol % Bis(2-chloroethyl)amine (on the Basis of the Molecular Weight of a Repeat Unit of Polyallylamine)

A mixture of a partially neutralized polyallylamine hydrochloride solution (see Example 6, 276.5 g), MgO (50 g, −325 mesh), and bis(2-chloroethyl)amine hydrochloride (9.46 g) was heated at 60° C. for 8 hours. A gel was formed after 5 minutes. After cooling to room temperature, the gel was broken into small pieces, washed with deionized water (3×4 L), and dried in a forced-air oven at 60° C. to afford 95.95 g.

### Example 33

### Preparation of Polyallylamine Crosslinked in the Presence of Magnesium Compound: 50 g PAA.HCl, 50 wt % MgO (on the Basis of the Weight of PAA.HCl), 20 mol % Bis(2-chloroethyl)amine (on the Basis of the Molecular Weight of a Repeat Unit of Polyallylamine)

A mixture of a partially neutralized polyallylamine hydrochloride solution (see Example 6, 276.5 g), MgO (25 g, −325 mesh), and bis(2-chloroethyl)amine hydrochloride (18.92 g) was heated at 60° C. for 8 hours. A gel was formed after 10 minutes. After cooling to room temperature, the gel was broken into small pieces, washed with deionized water (3×4 L), and dried in a forced-air oven at 60° C. to afford 63.73 g.

### Example 34

### Preparation of Polyallylamine Crosslinked in the Presence of Magnesium Compound: 50 g PAA.HCl, 100 wt % MgO (on the Basis of the Weight of PAA.HCl), 20 mol % Bis(2-chloroethyl)amine (on the Basis of the Molecular Weight of a Repeat Unit of Polyallylamine)

A mixture of a partially neutralized polyallylamine hydrochloride solution (see Example 6, 276.5 g), MgO (50 g, −325 mesh), and bis(2-chloroethyl)amine hydrochloride (18.92 g) was heated at 60° C. for 8 hours. A gel was formed after 5 minutes. After cooling to room temperature, the gel was broken into small pieces, washed with deionized water (3×4 L), and dried in a forced-air oven at 60° C. to afford 96.2 g.

### Example 35

### Preparation of Polyallylamine Crosslinked in the Presence of Magnesium Compound: 50 g PAA.HCl, 26.4 wt % MgO (on the Basis of the Weight of PAA.HCl), 12.5 mol % Epichlorohydrin (on the Basis of the Molecular Weight of a Repeat Unit of Polyallylamine)

To a partially neutralized polyallylamine hydrochloride solution (Example 6, 276.5 g) was added MgO (26.44 g, −325 mesh). After stirring for 10 minutes at room temperature, epichlorohydrin (5.23 mL) was added. A gel was formed. After curing at room temperature overnight, the gel was broken into small pieces, washed with deionized water, and dried in a forced-air oven at 60° C. to afford 64.64 g.

### Example 36

### Preparation of Polyallylamine Crosslinked in the Presence of Magnesium Compound: 50 g PAA.HCl, 53 wt % MgO (on the Basis of the Weight of PAA.HCl), 18 mol % Epichlorohydrin (on the Basis of the Molecular Weight of a Repeat Unit of Polyallylamine)

To a partially neutralized polyallylamine hydrochloride solution (see Example 6, 276.5 g) was added MgO (26.44 g, −325 mesh). After stirring for 10 minutes at room temperature, epichlorohydrin (7.53 mL) was added. A gel was formed. After curing at room temperature overnight, the gel was broken into small pieces, washed with deionized water, and dried in a forced-air oven at 60° C. to afford 65.96 g.

### Example 37

### Preparation of Polyallylamine Crosslinked in the Presence of Magnesium Compound: 10 g PAA.HCl, 18 wt % MgO (on the Basis of the Weight of PAA.HCl), 19.6 mol % Epichlorohydrin (on the Basis of the Molecular Weight of a Repeat Unit of Polyallylamine)

To a partially neutralized polyallylamine hydrochloride solution (see Example 6, 55.3 g) was added MgO (1.76 g, −325 mesh). After stirring for 1 hour at room temperature, epichlorohydrin (1.94 g) was added. A gel was formed. After curing at room temperature, the gel was broken into small pieces, washed with deionized water (3×1 L), and lyophilized to afford 8.35 g. Anal. Found: C, 50.28; H, 10.58; N, 16.13; Mg, 5.06.

### Example 38

### Preparation of Polyallylamine Crosslinked in the Presence of Magnesium Compound: 10 g PAA.HCl 18 wt % MgO (on the Basis of the Weight of PAA.HCl), 39.3 mol % Epichlorohydrin (on the Basis of the Molecular Weight of a Repeat Unit of Polyallylamine)

To a partially neutralized polyallylamine hydrochloride solution (see Example 6, 55.3 g) was added MgO (1.76 g, −325 mesh). After stirring for 1 hour at room temperature, epichlorohydrin (3.89 g) was added. A gel was formed. After curing at room temperature the gel was broken into small pieces, washed with deionized water (3×1 L), and lyophilized to afford 8.35 g. Anal. Found: C, 43.77; H, 9.18; N, 12.01; Mg, 3.94.

### Example 39

### Preparation of Polyallylamine Crosslinked in the Presence of Magnesium Compound: 20 g PAA.HCl, 35 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 110.6 g) was added MgO (7.06, −325 mesh). After stirring for 1 hour at room temperature, epichlorohydrin (2.91 g, 0.0315 mol, 15 mol %) was added. A gel was formed. After curing at room temperature, the gel was broken into small pieces, and washed with deionized water (3×2 L). The washed gel was split into two portions. One portion was lyophilized to give 10.8 g (Sample 39-#1). Anal. Found: Sample 39-#1, C, 36.32; H, 8.66; N, 12.00; Cl, 3.71; Mg, 13.56. The other portion was dried at 60° C. in a forced-air oven to give 10.87 g (Sample 3942). Anal. Found: Sample 39-#2, C, 39.50; H, 8.67; N, 12.97; Cl, 2.92; Mg, 14.56.

### Example 40

### Preparation of Polyallylamine Crosslinked in the Presence of Magnesium Compound: 20 g PAA.HCl, 35 wt % MgO (on the Basis of the Weight of PAA.HCl) 20 mol % Epichlorohydrin (on the Basis of the Molecular Weight of a Repeat Unit of Polyallylamine)

To a partially neutralized polyallylamine hydrochloride solution (see Example 6, 110.6 g, 20 g PAA.HCl, 0.21 mol) was added MgO (7.06, −325 mesh, 35.3% w/w). After stirring for 1 hour at room temperature, epichlorohydrin (3.88 g, 0.0419 mol, 0.2 equiv) was added. A gel was formed. After curing at room temperature the gel was broken into small pieces, and washed with deionized water (3×2 L). The washed gel was split into two portions. One portion was lyophilized to give 11.49 g (Sample 40-#1). Anal. Found: Sample 40-#1, C, 31.38; H, 7.82; N, 9.91; Cl, 4.31; Mg, 13.06. The other portion was dried at 60° C. in a forced-air oven to give 11.02 g (Sample 40-#2). Anal. Found: Sample 40-#1, C, 40.48; H, 8.98; N, 12.87; Cl, 3.85; Mg, 14.73.

### Example 41

### Preparation of Polyallylamine Crosslinked in the Presence of Magnesium Compound: 20 g PAA.HCl, 53 wt % MgO (on the Basis of the Weight of PAA.HCl), 10 mol % Epichlorohydrin (on the Basis of the Molecular Weight of a Repeat Unit of Polyallylamine)

To a partially neutralized polyallylamine hydrochloride solution (see Example 6, 110.6 g) was added MgO (10.60 g, nanopowder, 53% w/w). After stirring for 1 hour at room temperature, epichlorohydrin (1.94 g) was added. A gel was formed. After curing at room temperature, the gel was broken into small pieces, and washed with deionized water (5×2 L). The washed gel was dried at 60° C. in a forced-air oven to give 22.17 g. Anal. Found: C, 32.31; H, 6.70; N, 10.71; Cl, 2.41; Mg, 15.69.

### Example 42

### Preparation of Polyallylamine Crosslinked in the Presence of Magnesium Compound: 20 g PAA.HCl, 71 wt % MgO (on the Basis of the Weight of PAA.HCl), 10 mol % Epichlorohydrin (on the Basis of the Molecular Weight of a Repeat Unit of Polyallylamine)

To a partially neutralized polyallylamine hydrochloride solution (see Example 6, 110.6 g) was added MgO (14.14 g, nanopowder, 71% w/w). After stirring for 1 hour at room temperature, epichlorohydrin (1.94 g) was added. A gel was formed. After curing at room temperature, the gel was broken into small pieces, and washed with deionized water (5×2 L). The washed gel was dried at 60° C. in a forced-air oven to give 24 g. Anal. Found: C, 36.12; H, 8.12; N, 11.97; Cl, 1.23; Mg, 17.36.

### Example 43

### Preparation of Polyallylamine Crosslinked in the Presence of Magnesium Compound: 50 g PAA.HCl, 53 wt % MgO (on the Basis of the Weight of PAA.HCl), 9.8 mol % 1,4-Butanediol Diglycidyl Ether (on the Basis of the Molecular Weight of a Repeat Unit of Polyallylamine)

To a partially neutralized polyallylamine hydrochloride solution (see Example 6, 276.5 g) was added MgO (26.44 g). After stirring for 1 hour at room temperature, 1,4-butanediol diglycidyl ether (9.93 mL) was added. A gel was formed within a couple of minutes. After curing at room temperature over 3 nights, the gel was broken into small pieces and suspended into deionized water (4 L). After stirring for 20 minutes, the suspension was filtered. The filtered polymer was washed with deionized water (2×4 L). The filtered polymer was dried in a forced-air oven at 60° C. to afford 73.16 g. Anal. Found: C, 34.53; H, 7.80; N, 9.69; Cl, 3.00; Mg, 18.29.

### Example 44

### Preparation of Polyallylamine Crosslinked in the Presence of Magnesium Compound: 50 g PAA.HCl, 53 wt % MgO (on the Basis of the Weight of PAA.HCl), 9.8 mol % 1,2-Dibromoethane (on the Basis of the Molecular Weight of a Repeat Unit of Polyallylamine)

To a partially neutralized polyallylamine hydrochloride solution (see Example 6, 276.5 g) was added MgO (26.44 g). After stirring for 30 minutes at room temperature, 1,2-dibromoethane (4.52 mL) was added. The mixture was heated to 60° C. overnight. A gel was formed within a couple of minutes. After curing at room temperature over 3 nights, the gel was broken into small pieces and suspended into deionized water (4 L). After stirring for 20 minutes, the suspension was filtered. The filtered polymer was washed with deionized water (2×4 L). The filtered polymer was dried in a forced-air oven at 60° C. to afford 63.84 g. Anal. Found: C, 34.36; H, 7.82; N, 11.76; Cl, 0.87; Br, 0.79; Mg, 19.21.

### Example 45

### Preparation of Polyallylamine Crosslinked in the Presence of Magnesium Compound: 50 g PAA.HCl, 35 wt % MgO (on the Basis of the Weight of PAA.HCl) 15 mol % Epichlorohydrin (on the Basis of the Molecular Weight of a Repeat Unit of Polyallylamine)

To a partially neutralized polyallylamine hydrochloride solution (see Example 6, 276.5 g) was added MgO (17.65 g). After stirring for 1 hour at room temperature, epichlorohydrin (6.22 mL) was added. A gel was formed within 30 minutes. After curing at room temperature over 3 nights, the gel was broken into small pieces and suspended into deionized water (4 L). After stirring for 20 minutes, the suspension was filtered. The filtered polymer was washed with deionized water (2×4 L). The filtered polymer was dried in a forced-air oven at 60° C. to afford 56.21 g.

### Example 46

### Preparation of Polyallylamine Crosslinked in the Presence of Magnesium Compound: 50 g PAA.HCl, 53 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 276.5 g) was added MgO (26.44 g). After stirring for 1 hour at room temperature, epichlorohydrin (6.22 mL) was added. A gel was formed within 30 minutes. After curing at room temperature over 3 nights, the gel was broken into small pieces and suspended into deionized water (4 L). After stirring for 20 minutes, the suspension was filtered. The filtered polymer was washed with deionized water (2×4 L each wash). The filtered polymer was dried in a forced-air oven at 60° C. to afford 64.04 g.

### Example 47

### Preparation of Polyallylamine Crosslinked in the Presence of Magnesium Compound: 50 g PAA.HCl, 70.5 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 276.5 g) was added MgO (35.25 g). After stirring for 1 hour at room temperature, epichlorohydrin (6.22 mL) was added. A gel was formed within 30 minutes. After curing at room temperature over 3 nights, the gel was broken into small pieces and suspended into deionized water (4 L). After stirring for 20 minutes, the suspension was filtered. The filtered polymer was washed with deionized water (2×4 L). The filtered polymer was dried in a forced-air oven at 60° C. to afford 79.48 g.

### Example 48

### Preparation of Polyallylamine Crosslinked in the Presence of Magnesium Compound: 50 g PAA.HCl, 100 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 276.5 g) was added MgO (35.25 g). After stirring for 1 hour at room temperature, epichlorohydrin (6.22 mL) was added. A gel was formed within 30 minutes. After curing at room temperature over 3 nights, the gel was broken into small pieces and suspended into deionized water (4 L). After stirring for 20 minutes, the suspension was filtered. The filtered polymer was washed with deionized water (2×4 L). The filtered polymer was dried in a forced-air oven at 60° C. to afford 97.71 g.

### Example 49

### Preparation of Polyallylamine Crosslinked in the Presence of Magnesium Compound: 50 g PAA.HCl, 120 wt % MgO (on the Basis of the Weight of PAA.HCl), 15 mol % Epichlorohydrin (on the Basis of the Molecular Weight of a Repeat Unit of Polyallylamine)

To a partially neutralized polyallylamine hydrochloride solution (see Example 6, 276.5 g) was added MgO (35.25 g). After stirring for 1 hour at room temperature, epichlorohydrin (6.22 mL) was added. A gel was formed within 30 minutes. After curing at room temperature over 3 nights, the gel was broken into small pieces and suspended into deionized water (4 L). After stirring for 20 minutes, the suspension was filtered. The filtered polymer was washed with deionized water (2×4 L each wash). The filtered polymer was dried in a forced-air oven at 60° C. to afford 108.9 g.

### Example 50

### Preparation of Polyallylamine Crosslinked in the Presence of Magnesium Compound: Mg(OH)₂

To a partially neutralized polyallylamine hydrochloride solution (see Example 6, 110.6 g) was added Mg(OH)₂ (12.25 g). After stirring for 10 minutes at room temperature, epichlorohydrin (0.985 mL) was added. A gel was formed within 1 hour. After curing at room temperature over 3 nights, the gel was broken into small pieces and suspended into deionized water (4 L). After stirring for 20 minutes, the suspension was filtered. The filtered polymer was washed with deionized water (1×4 L). The filtered polymer was lyophilized to afford 23.81 g. Anal. Found: C, 31.55; H, 8.49; N, 11.27; Cl, 6.89; Mg, 20.06.

### Example 51

### Preparation of Polyallylamine Crosslinked in the Presence of Magnesium Compound: MgCl₂

To a partially neutralized polyallylamine hydrochloride solution (see Example 6, 110.6 g) was added MgCl₂ (20 g). After stirring for 10 minutes at room temperature, epichlorohydrin (0.985 mL) was added. A gel was formed within 1 hour. After curing at room temperature over 3 nights, the gel was broken into small pieces and suspended into a 70:30 solution of isopropanol and deionized water (4 L). After stirring for 20 minutes, the suspension was filtered. The filtered polymer was washed three with a 70:30 solution of isopropanol and deionized water (3×4 L). The filtered polymer was lyophilized to afford 18.04 g. Anal. Found: C, 38.62; H, 9.67; N, 13.53; Cl, 20.98; Mg, 3.54.

### Example 52

### Preparation of Polyallylamine Crosslinked in the Presence of Magnesium Compound: 50 & PAA.HCl, 150 wt % Mg(OEt)₂(on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 276.5 g) was added Mg(OEt)2 (75.07 g). After stirring for 1 hour at room temperature, epichlorohydrin (4.10 mL) was added. A gel was formed within 45 minutes. After curing at room temperature overnights the gel was broken into small pieces. Half of the gel was dried in a forced-air oven at 60° C. to afford 50.46 g (Example 52-#1). The other half of the initial gel was suspended into deionized water (4 L). After stirring for 20 minutes, the suspension was filtered. The filtered polymer was washed with deionized water (1×4 L). This washed and filtered polymer was dried in a forced-air oven at 60° C. to afford 29.91 g (Example 52-#2). Anal. Found: Example 52-#2, C, 19.69; H, 6.19; N, 6.43; Cl, 6.73; Mg, 16.39. A 50 g portion of Example 52-#1 was ground and sieved through a −80 mesh screen and suspended into deionized water (4 L). After stirring for 15 minutes, the suspension was filtered. The filtered polymer was washed twice more with deionized water (4 L each wash) and was dried in a forced-air oven at 60° C. to afford 29.91 g (Example 52-#1). Anal. Found: Example 52-#1, C, 30.46; H, 7.93; N, 10.07; Cl, 2.01; Mg, 17.97.

### Example 53

### Preparation of Polydiallylamine Crosslinked in the Presence of Magnesium Compound MgO

To a partially neutralized solution of polydiallylamine hydrochloride (76 g, pH 10.13 and equivalent to 26.3% (w/w) of poly(diallylamine) hydrochloride) was added MgO (1.51 g). After stirring for 20 minutes at room temperature, epichlorohydrin (2.11 mL) was added. A gel was formed within 20 minutes. After curing at room temperature overnight, the gel was broken into small pieces and suspended into deionized water (4 L). After stirring for 20 minutes, the suspension was filtered. The filtered polymer was suspended again in deionized water (4 L), stirred for 20 minutes, and filtered. The filtered polymer was lyophilized to afford 17.8 g.

### Example 54

### Preparation of Polyethylenimine Crosslinked in the Presence of Magnesium Compound: MgO

To a solution of polyethylenimine (20 g, Mw 25000, water free) in deionized water (80 g) was added MgO (4.64 g). After stirring for 20 minutes at room temperature, epichlorohydrin (3.24 mL) was added. After curing at room temperature overnight, the gel was broken into small pieces and suspended into deionized water (4 L). After stirring for 20 minutes, the suspension was filtered. The filtered polymer was suspended again in deionized water (4 L), stirred for 20 minutes, and filtered. The filtered polymer was dried in a forced-air oven at 60° C. to afford 24.61 g. The dried solid was suspended in deionized water (4 L). Concentrated HCl was added until the suspension had pH 1. After filtering the polymer was dried in a forced-air oven at 60° C. to afford 29.79 g.

### Example 55

### Preparation of Poly(Vinylamine) Crosslinked in the Presence of Magnesium Compound: MgO

To a solution of poly(vinylamine) hydrochloride (20 g) in deionized water (80 mL) was added MgO (2.52 g). After stirring for 1 hour at room temperature, 50% NaOH (8.57 g) was added followed by epichlorohydrin (1.76 mL). After curing at room temperature overnight, the gel was broken into small pieces and suspended into deionized water (4 L). After stirring for 20 minutes, the suspension was filtered. The filtered polymer was suspended again in deionized water (4 L), stirred for 20 minutes, and filtered. The filtered polymer was dried in a forced-air oven at 60° C. to afford 14.3 g. Anal. Found: C, 45.11; H, 8.88; N, 19.68; Mg, 1.39.

### Example 56

### Preparation of Admixture of Crosslinked Poly[4-{(tris(2-aminoethyl)amino)methyl}styrene] and MgO

To a vial was added finely ground 9 mol % epichlorohydrin-crosslinked poly[4-{(tris(2-aminoethyl)amino)methyl}styrene] (10.8 g) followed by MgO (4.2 g, −325 mesh). The vial was shaken by hand for approximately 5 minutes.

### Example 57

### Preparation of Epichlorohydrin-Crosslinked Poly[4-{(bis(3-aminopropyl)amino)methyl}styrene]

To a solution of poly[4-{(bis(3-aminopropyl)amino)methyl}styrene] hydrochloride (13.25 g) in deionized water (53 g) was added NaOH (6.46 g of a 50% aqueous solution) to a solution pH 10. Epichlorohydrin (0.261 mL) was then added. The solution was stirred with an overhead stirrer at room temperature until it gelled, and the gel was allowed to cure at room temperature. After curing at room temperature, the gel was broken into small pieces and suspended into a 70:30 solution of isopropanol and deionized water (4 L). After stirring for 20 minutes, the suspension was filtered. The filtered polymer was dried in a forced-air oven at 60° C. to afford 12.01 g.

### Example 58

### Preparation of Admixture of Epichlorohydrin-Crosslinked Poly[4-{(bis(3-aminopropyl)amino)methyl}styrene] and MgO

To a vial was added finely ground epichlorohydrin crosslinked poly[4-{(bis(3-aminopropyl)amino)methyl}styrene] (3.5 g of Example 64) followed by MgO (1.4 g, −325 mesh). The vial was shaken by hand for approximately 5 minutes.

### Example 59

### Preparation of Ethylenebisacrylamide-Crosslinked Poly[4-{(tris(3-aminoethyl)amino)methyl}styrene]

A stirred solution of 4-{(tris(2-aminoethyl)amino)methyl}styrene (15 g), deionized water (35 mL), N,N-ethylenebisacrylamide (0.5 g), and 2,2′-azobisamidinopropane dihydrochloride (0.75 g of a 20% aqueous solution) was heated at 60° C. for 18 hours under a nitrogen atmosphere. The solution became a gel within 30 minutes. After cooling to room temperature the gel was broken into small pieces and suspended into methanol (1 L). After stirring for 15 minutes, the suspension was filtered. The filtered polymer was washed with methanol (12×1 L). The filtered polymer was then suspended into deionized water (1 L). After stirring for 15 minutes, the suspension was filtered. The filtered polymer was then washed with water (2×1 L each wash). The pH of the final aqueous suspension was adjusted to 7 with the addition of concentrated HCl. The filtered polymer was dried in a forced-air oven at 60° C. to afford 12.56 g.

### Example 60

### Preparation of Admixture of Ethylenebisacrylamide-Crosslinked Poly[4-{(tris(3-aminoethyl)amino)methyl}styrene] and MgO

To a vial was added finely ground ethylenebisacrylamide cross linked poly[4-{(tris(3-aminoethyl)amino)methyl}styrene] (2.5 g, Example 66) followed by MgO (1 g, −325 mesh). The vial was shaken by hand for approximately 5 minutes.

### Example 61

### Preparation of Ethylenebisacrylamide-Crosslinked Poly[4-{(tris(3-aminoethyl)amino)methyl}styrene]

A stirred solution of 4-{(tris(2-aminoethyl)amino)methyl}styrene (15 g), deionized water (35 mL), N,N-ethylenebisacrylamide (1 g), and 2,2′-azobisamidinopropane dihydrochloride (0.75 g of a 20% aqueous solution) was heated at 60° C. for 18 hours under a nitrogen atmosphere. The solution became a gel within 30 min. After cooling to room temperature the gel was broken into small pieces and suspended into methanol (1 L). After stirring for 15 minutes, the suspension was filtered. The filtered polymer was washed times with methanol (2×1 L). The filtered polymer was then suspended into deionized water (1 L). After stirring for 15 minutes, the suspension was filtered. The filtered polymer was washed similarly with water (2×1 L). The pH of the final aqueous suspension was adjusted to 7 with the addition of concentrated HCl. The filtered polymer was dried in a forced-air oven at 60° C. to afford 13.98 g.

### Example 62

### Preparation of Admixture of Ethylenebisacrylamide-Crosslinked Poly[4-[(tris(3-aminoethyl)amino)methyl]styrene] and MgO

To a vial was added finely ground ethylenebisacrylamide-crosslinked poly[4-{(tris(3-aminoethyl)amino)methyl}styrene] (2.5 g of Example 68) followed by MgO (1 g, −325 mesh). The vial was shaken by hand for approximately 5 minutes.

### Example 63

### Preparation of Crosslinked Poly[4-{(tris(3-aminoethyl)amino)methyl}styrene]

A stirred solution of 4-{(tris(2-aminoethyl)amino)methyl}styrene (15 g), deionized water (35 mL), cross linker N,N′-bis[(4-vinyl)benzyl]ethylenediamine (0.83 g, Example 70), and 2,2′-azobisamidinopropane dihydrochloride (0.75 g of a 20% aqueous solution) was heated at 60° C. for 18 hours under a nitrogen atmosphere. The solution became a gel within 4 hours. After cooling to room temperature the gel was broken into small pieces and suspended into methanol (2 L). After stirring for 15 minutes the suspension was filtered. The filtered polymer was washed similarly two more times with methanol (2 L each wash). The filtered polymer was then suspended into deionized water (2 L). After stirring for 15 minutes the suspension was filtered. The filtered polymer was washed similarly two more times with water (2 L each wash). The pH of the final aqueous suspension was adjusted to 7 with the addition of concentrated HCl. The filtered polymer was dried in a forced-air oven at 60° C. to afford 13 g.

### Example 64

### Preparation of Admixture of Crosslinked Poly[4-{(tris(3-aminoethyl)amino)methyl}styrene] and MgO

To a vial was added finely ground cross linked poly[4-{(tris(3-aminoethyl)amino)methyl}styrene] (2.5 g, Example 70) followed by MgO (1 g, −325 mesh). The vial was shaken by hand for approximately 5 minutes.

### Example 65

### Preparation of Poly[4-{(tris(2-aminoethyl)amino)methyl}styrene] Crosslinked in the Presence of Magnesium Compound: MgO, 70 wt % on the Basis of the Weight of Poly[4-{(tris(2-aminoethyl)amino)methyl}styrene]

To a solution of poly [4-{(tris(2-aminoethyl)amino)methyl}styrene] (10 g) in deionized water (53 mL) cooled in an ice-water bath was slowly added 50% NaOH until the solution had pH 10.5. To this solution was added MgO (7 g, −325 mesh) and stirred for 1 hour. Epichlorohydrin (0.204 g) was added and the mixture was stirred until a gel formed (2 h). After curing at room temperature, the gel was broken into small pieces and suspended into deionized water (2 L). After stirring for 50 minutes, the suspension was filtered. The filtered polymer was washed with deionized water (2×2 L). The filtered polymer was lyophilized to afford 10.3 g. Anal. Found: C, 23.42; H, 5.28; N, 6.62; Mg, 29.63.

### Example 66

### Preparation of Poly [4-{(tris(2-aminoethyl)amino)methyl}styrene] Crosslinked in the Presence of Magnesium Compound: MgO 50 wt % on the basis of the weight of poly [4-{(tris(2-aminoethyl)amino)methyl}styrene]

To a solution of poly [4-{(tris(2-aminoethyl)amino)methyl}styrene] (10 g) in deionized water (53 mL) cooled in an ice-water bath was slowly added 50% NaOH until the solution had pH 10.5. To this solution was added MgO (5 g, −325 mesh) and stirred for 1 hour. Epichlorohydrin (0.204 g) was added and the mixture was stirred until a gel formed (about 2 hours). After curing at room temperature, the gel was broken into small pieces and suspended into deionized water (2 L). After stirring for 50 minutes, the suspension was filtered. The filtered polymer was washed with deionized water (2×2 L). The filtered polymer was lyophilized to afford 7.4 g. Anal. Found: C, 33.02; H, 6.45; N, 9.41; Mg, 29.30.

### Example 67

### Preparation of Poly [4-{(tris(2-aminoethyl)amino)methyl}styrene] Crosslinked in the Presence of Magnesium Compound: MgO, 30 wt % on the basis of the weight of poly [4-{(tris(2-aminoethyl)amino)methyl}styrene]

To a solution of poly [4-{(tris(2-aminoethyl)amino)methyl}styrene] (10 g) in deionized water (53 mL) cooled in an ice-water bath was slowly added 50% NaOH until the solution had pH 10.5. To this solution was added MgO (3 g, −325 mesh) and stirred for 1 hour. Epichlorohydrin (0.204 g) was added and the mixture was stirred until a gel formed (about 3 hours). After curing at room temperature the gel was broken into small pieces, washed with water, and lyophilized.

### Example 68

### Preparation of Polyallylamine Crosslinked in the Presence of Magnesium Compound: MgO

To a solution of poly(allylamine) free base (20.25 g) in anhydrous methanol (80 g) was added MgO (8.3 g, −325 mesh). After stirring for 20 minutes, epichlorohydrin (2.5 mL) was added. The mixture was stirred for 2 hours at room temperature and then heated at 60° C. overnight. A gel formed after heating for 90 minutes. The gel was broken into small pieces. Half of the gel was concentrated on a rotary evaporator and dried in a vacuum oven at 60° C. (Sample 68-#1). The other half was suspended in anhydrous MeOH (300 mL), stirred, filtered, and dried in a vacuum oven at 60° C. (Sample 68-#2).

### Example 69

### Preparation of Polyallylamine Crosslinked in the Presence of Magnesium Compound: Mg(OH)₂

To a solution of polyallylamine free base (10 g) in anhydrous methanol (40 g) was added Mg(OH)₂ (6 g). After stirring for 30 minutes, epichlorohydrin (1.24 mL) was added. The mixture was heated at 60° C. overnight. A gel was formed after heating for 1.5-2 hours. The gel was broken into small pieces. Half of the gel was concentrated on a rotary evaporator and dried in a vacuum oven at 60° C. (Sample 69-1). Anal. Found for Sample 69-#1: C, 32.83; H, 7.81; N, 10.58; Cl, 4.06; Mg, 13.86. The other half was suspended in anhydrous MeOH (300 mL), stirred, filtered, and dried in a vacuum oven at 60° C. (Sample 69-#2). Anal. Found for Sample 69-#2: C, 29.10; H, 7.06; N, 9.17; Cl, 6.05; Mg, 18.54.

### Example 70

### Preparation of Polyallylamine Crosslinked in the Presence of Magnesium Compound: 69.6 g PAA.HCl 53 wt % MgO (on the Basis of the Weight of PAA.HCl), 9.8 mol % Epichlorohydrin (on the Basis of the Molecular Weight of a Repeat Unit of Polyallylamine)

To a partially neutralized polyallylamine hydrochloride solution (see Example 6, 385 g) was added MgO (36.85 g). After stirring for 2 minutes at room temperature, epichlorohydrin (5.71 mL) was added. A gel was formed. After curing at room temperature over 1 hour, the gel was broken into small pieces and suspended into deionized water (4 L). After stirring for 5 minutes, the suspension was filtered. The filtered polymer was washed with deionized water (2×4 L). The filetered polymer was lyophilized to afford 88.29 g.

### Example 71

### Analysis of Contents of Components of Materials

Magnesium contents of the examples above were analyzed by ICP-OES (Inductively Coupled Plasma Optical Emission Spectroscopy). Selected results are summarized in Table 1. Percent chloride was analyzed by titration with silver nitrate.

Percent loss-on-drying (LOD) was determined by TGA (thermogravimetic analysis) (see Tables 2 and 3). For LOD, an oven was programmed to increase the oven temperature 10 degrees per minute to 85° C., hold for 60 minutes, and then 10 degrees per minute to 300° C. The percent weight change for LOD determined between 0 and 65 minutes.

TABLE 1
Magnesium content as determined by ICP-OES.

| | Mg content (wt % of the combined |
|---|---|
| Sample Number | weight of Mg ion and polymer) |
| Example 15 | 24.1 |
| Example 16 | 13.3 |
| Example 19 | 0.3 |
| Example 37 | 4.5 |
| Example 38 | 3.4 |
| Example 17 | 5.0 |
| Example 7-#1 | 21.3 |
| Example 7-#2 | 25.4 |
| Example 8-#1 | 19.2 |
| Example 8-#2 | 24.6 |
| Example 22-#1 | 13.4 |
| Example 22-#2 | 15.7 |
| Example 23-#1 | 12.4 |
| Example 23-#2 | 16.5 |
| Example 66 | 21.4 |
| Example 10 | 18.1 |
| Example 41 | 10.4 |
| Example 50 | 18.9 |
| Example 51 | 2.2 |
| Example 52-#1 | 18.7 |
| Example 52-#2 | 17.4 |
| Example 20-#1 | 19.4 |
| Example 20-#2 | 18.1 |
| Example 21-#1 | 18.3 |
| Example 21-#2 | 17.7 |
| Example 9 | 18.4 |
| Example 24 | 16.0 |
| Example 25 | 15.9 |
| Example 26 | 13.6 |
| Example 70 | 16.0 |
| Example 27 | 17.1 |
| Example 28 | 13.7 |
| Example 11 | 20.5 |
| Example 29 | 23.5 |
| Example 12 | 27.3 |
| Example 13 | 31.4 |
| Example 14 | 5.7 |

TABLE 2
Characterization of New Phosphate Binder Samples

Test Sample Name

| Test Name | Example 10 | Example 9-#1 |
|---|---|---|
| LOD by TGA | 5.85 | 6.50 |
| (%) | | |
| DSC (Tg onset | 65.09° C. | 66.82° C. |
| Temp.) | | |
| FTIR (cm⁻¹) | 3695 (OH str.) | 3695 (OH str.) |
| | 2911 (C—H str.) | 2909 (C—H str.) |
| | 1567 (NH | 1569 (NH |
| | bending) | bending) |
| Mg content (%) | 18.1 | 18.4 |
| pH | 9.79 | 9.83 |
| Titrable Amines | 22.9 | 23.2 |
| (mmol/g) | | |
| Chloride (%) | 5.2 | 3.96 |
| Elemental (%) | C, 28.58 | C, 28.58 |
| | H, 7.67 | H, 7.69 |
| | N, 9.60 | N, 9.54 |
| | Cl, 4.92 | Cl, 3.60 |
| Soluble | 0.23 | 0.28 |
| Oligomers (%) | | |
| Phosphate | 4.67 | 4.31 |
| Binding (mmol PO₄/g) | | |
| Allylamine | 3.50 | 2.59 |
| (ppm) | | |

### Example 72

### In Vivo Phosphate Binding: Effects of Polyamine-Magnesium Compounds for Reducing Urinary Phosphate Levels

House male Sprague Dawley (SD) rats were used for the experiments. The rats were placed singly in wire-bottom cages, fed with Purina 5002 diet, and allowed to acclimate for at least 5 days prior to experimental use.

To establish baseline phosphorus excretion, the rats were placed in metabolic cages for 48 hours. Their urine was collected and its phosphorus content analyzed with a Hitachi analyzer to determine phosphorus excretion in mg/day. Any rats with outlying values were excluded; and the remainder of the rats was distributed into groups.

Purina 5002 was used as the standard diet. The compound being tested was mixed with Purina 5002 to result in a final concentration by weight as noted in the table. Cellulose at 0.5% by weight was used as a negative control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. A change in phosphorus excretion relative to baseline and cellulose negative control was calculated using Excel program. A summary of comparison of the amounts of urinary phosphate obtained from the test rats is shown in the Table 3 below.

TABLE 3
Amounts of Urinary Phosphate in Tested SD Rats

| | | % Urinary |
|---|---|---|
| | | Phosphate |
| | | Relative to that of |
| Treatment | % of Diet | Control Animals |
| Example 15 | 0.50 | 27.6 |
| MgO | 0.25 | 69.3 |
| Example 38 | 0.25 | 109.8 |
| Example 55 | 0.25 | 89.6 |
| Example 16 | 0.25 | 56.1 |
| Example 16 | 0.15 | 72.4 |
| Sevelamer HCl/MgO | 0.25 | 59.6 |
| MgO | 0.20 | 61.6 |
| MgO | 0.30 | 54.8 |
| MgO | 0.40 | 26.1 |
| Sevelamer HCl/MgO | 0.35 | 63.3 |
| Sevelamer HCl/MgO | 0.40 | 60.8 |
| Sevelamer HCl/MgO | 0.45 | 49.6 |
| Example 15 | 0.25 | 56.1 |
| Example 65 | 0.25 | 68.9 |
| Example 65 | 0.40 | 39.8 |
| Example 8-#1 | 0.25 | 56.3 |
| Example 8-#2 | 0.25 | 55.8 |
| Example 22-#2 | 0.25 | 73.7 |
| Example 23-#2 | 0.25 | 62.4 |
| MgO | 1.00 | 3.6 |
| MgCl2 | 2.38 | 57.0 |
| A mixture of Example 7-#1 and 7-#2 | 2.60 | 1.0 |
| Example 39-#2 | 0.25 | 89.0 |
| Example 7-#1 | 0.25 | 84.3 |
| Example 41 | 0.25 | 65.4 |
| Example 42 | 0.25 | 66.8 |
| Example 67 | 0.25 | 55.7 |
| Example 66 | 0.30 | 49.7 |
| Example 59 | 0.50 | 56.6 |
| Example 61 | 0.50 | 63.0 |
| Example 63 | 0.50 | 60.9 |
| Example 59/MgO | 0.25 | 73.7 |
| Example 61/MgO | 0.25 | 61.7 |
| Example 63 | 0.25 | 66.3 |
| Example 57 | 0.50 | 61.2 |
| Example 58 | 0.30 | 66.4 |
| Example 52 | 0.25 | 57.7 |
| Example 10 | 0.50 | 54.9 |
| Example 10 | 0.35 | 70.4 |
| Example 10 | 0.25 | 68.6 |
| Example 10 | 0.15 | 77.0 |
| Example 10 | 0.50 | 30.5 |
| Example 10 | 0.35 | 44.7 |
| Example 10 | 0.25 | 52.2 |
| Example 10 | 0.25 | 57.8 |
| Example 10 | 0.25 | 65.8 |
| Example 56 | 0.25 | 73.6 |
| Example 9-#1 | 0.25 | 64.6 |
| Example 9-#2 | 0.25 | 71.2 |
| Example 24 | 0.25 | 66.7 |
| Example 25 | 0.25 | 88.4 |
| Example 26 | 0.25 | 84.9 |
| Example 68-#1 | 0.25 | 73.9 |
| Example 68-#2 | 0.25 | 62.5 |
| Example 50 | 0.25 | 71.6 |
| Example 69-#1 | 0.25 | 78.2 |
| Example 69-#2 | 0.25 | 70.2 |

### Example 73

### Magnesium Uptake in Rats Treated with Sevelamer

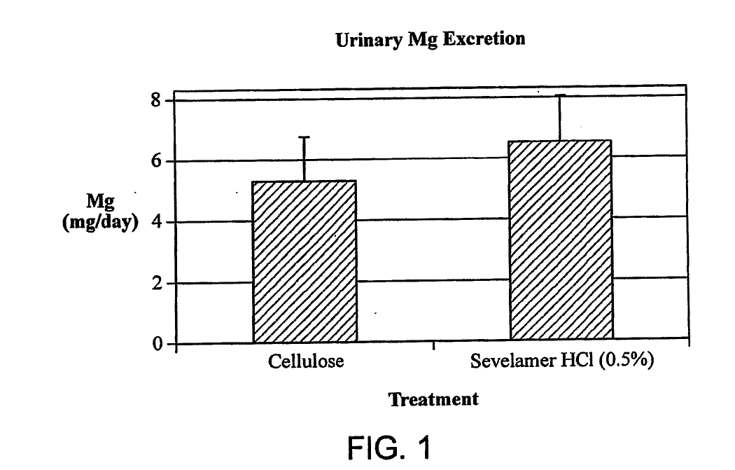

Magnesium uptake by rats treated with sevelamer hydrochloride alone (72 rats) and cellulose as a control (66 rats) was quantitatively estimated by the analysis of urine samples of tested rats in a manner similar to the phosphate analysis in Example 72. For the test rats, Purina 5002 was used as a standard diet. Sevelmer hydrochloride and cellulose were each independently mixed with Purina 5002 to result in a final concentration by weight as noted in FIG. 1. Cellulose at 0.5% by weight was used as a negative control.

For each rat, 200 g of diet was prepared. Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. For analysis, the urine samples were diluted with 1N HCl in a volume ratio of 1:2 (acid to urine), and the magnesium content of the urine samples was estimated by Hitachi 912 clinical chemistry analyzer. A change in magnesium excretion relative to the cellulose control was used to quantify magnesium uptake of the rats treated with sevelamer hydrochloride.

The results of magnesium uptake in rats (total 66) treated with 0.5% of diet of sevelamer hydrochloride and rats (total 72) treated with cellulose are shown in FIG. 1. As can be seen in FIG. 1, a slight increase in magnesium uptake was observed with the sevelamer hydrochloride treatment.

### Example 74

### Magnesium Uptake in Rats Treated with Polyallylamine Crosslinked in the Presence of a Magnesium Compound (PAA/Mg)

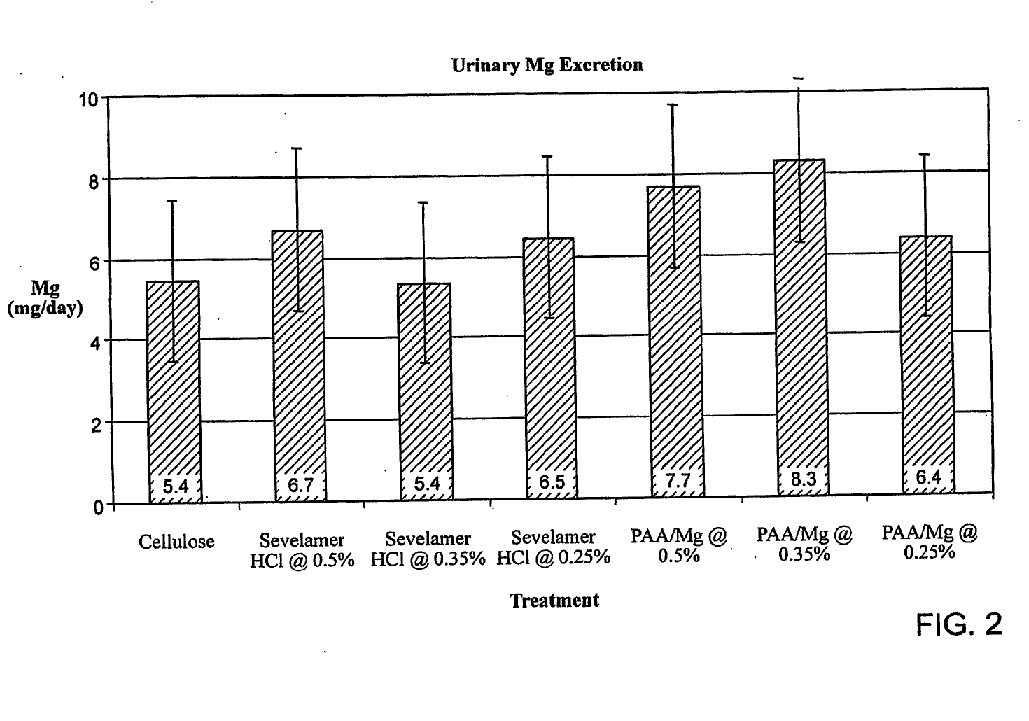

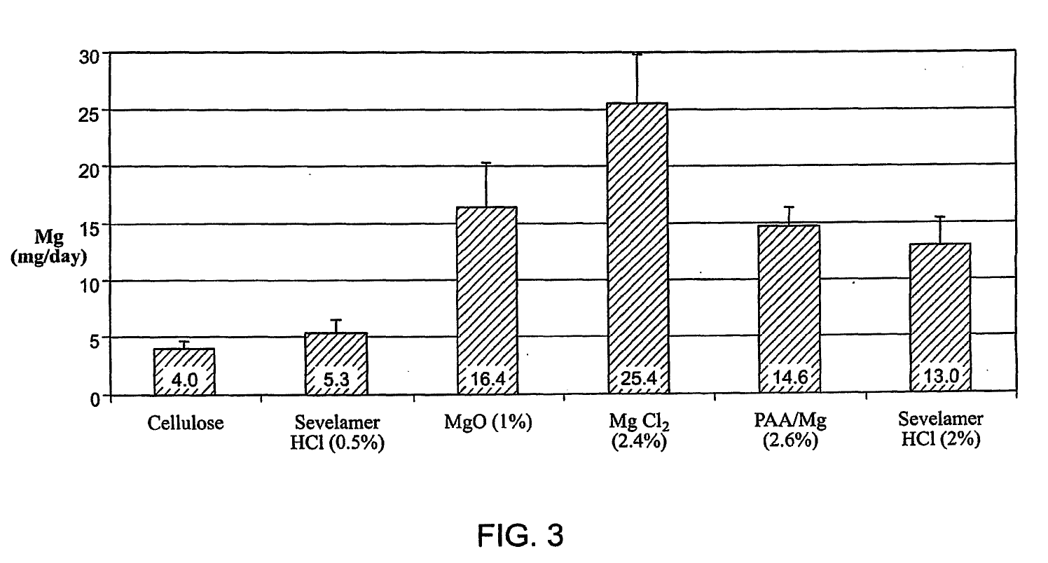

Magnesium uptake by rats treated with polyallylamine-magnesium compounds (PAA/Mg) of Examples 10 (FIG. 2) and 7 (FIG. 3) was quantitatively estimated by the analysis of urine samples in a manner similar to the phosphate analysis in Example 72. For the test rats, Purina 5002 was used as a standard diet. Cellulose, sevelamer hydrochloride and polyallylamine-magnesium compounds were each independently mixed with Purina 5002 to result in a final concentration by weight as noted in FIGS. 2 and 3. Cellulose at 0.5% by weight was used as a negative control. For each rat, 200 g of diet was prepared.

Each rat was weighed and placed on the standard diet. After 4 days the standard diet was replaced with the treatment diet (or control diet for the control group). On days 5 and 6, urine samples from the rats at 24 hours (+/−30 minutes) were collected and analyzed. The test rats were again weighed, and any weight loss or gain was calculated. Any remaining food was also weighed to calculate the amount of food consumed per day. For analysis, the urine samples were diluted with 1N HCl in a volume ratio of 1:2 (acid to urine), and the magnesium content of the urine samples was estimated by Hitachi 912 clinical chemistry analyzer. A change in magnesium excretion relative to the cellulose control was used to quantify magnesium uptake of the rats treated with polyallylamine-magnesium compounds and sevelamer hydrochloride.

The results of magnesium uptake in rats associated with PAA/Mg treatment and other control treatments, i.e., sevelamer hydrochloride, MgO, and MgCl₂ treatments, are shown in FIGS. 2 and 3. Shown in FIG. 2 are magnesium contents in urine samples of the test rats treated with cellulose as a control, sevelamer hydrochloride at 0.5%, 0.35% and 0.25% diet, and polyallylamine-magnesium compound (PAA/Mg) of Example 7 (a mixture of Example 7-#1 and Example 7-#2) at 0.5%, 0.35% and 0.25% diet. Shown in FIG. 3 are magnesium contents in urine samples of the test rats treated with cellulose as a control, sevelamer hydrochloride at 0.5% diet, MgO at 1% diet, MgCl₂ in 2.4% diet, polyallylamine-magnesium compound (PAA/Mg) of Example 10 at 2.6% diet, and sevelamer hydrochloride at 2% diet. As shown in FIG. 2, in vivo magnesium uptake in rats treated with 0.25% diet (low phosphate-diet) of PAA/Mg was not much higher than that in rats treated with 0.25% (low phosphate-diet) diet of sevelamer hydrochloride. A similar result was also observed when tested rats were under a high-phosphate diet, i.e., 2.6% of PAA/Mg and 2% of sevelamer hydrochloride (see FIG. 3). That is, surprisingly, PAA/Mg phosphate binder did not raise magnesium uptake any more than did sevelamer hydrochloride alone despite the presence of the magnesium compound, such as MgO.

While this invention has been particularly shown and described with references to preferred embodiments thereof, it will be understood by those skilled in the art that various changes in form and details may be made therein without departing from the scope of the invention encompassed by the appended claims.

1. A pharmaceutical composition, comprising:
a) an aliphatic amine polymer or a pharmaceutically acceptable salt thereof; and
b) a pharmaceutically acceptable magnesium compound comprising a magnesium ion,
wherein the magnesium ion is 5-35% by anhydrous weight of the pharmaceutical composition.

2. The pharmaceutical composition of claim 1, wherein the magnesium compound is selected from the group consisting of magnesium oxide, magnesium hydroxide, magnesium carbonate, magnesium formate and a combination thereof.

3. (canceled)

4. The pharmaceutical composition of claim 1, wherein the aliphatic amine polymer comprises one or more repeat units represented by a structural formula selected from: 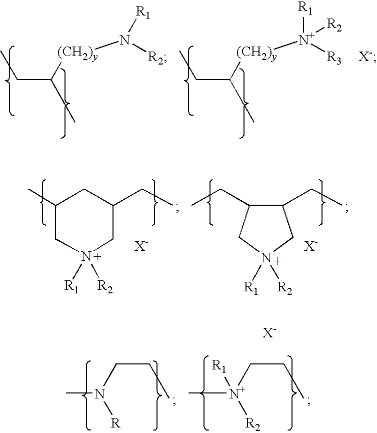
or a salt thereof, wherein:
y and z are independently zero or an integer from one to ten;
R, R₁, R₂ and R₃, independently, are H, a substituted or unsubstituted alkyl group or an aryl group; and
X⁻ is an exchangeable negatively charged counterion.

5-7. (canceled)

8. The pharmaceutical composition of claim 4, wherein the aliphatic amine polymer is crosslinked polyallylamine.

9. The pharmaceutical composition of claim 8, wherein the polyallylamine polymer is sevelamer.

10. The pharmaceutical composition of claim 9, wherein the polyallylamine polymer is a chloride salt of sevelamer, a carbonate salt of sevelamer or a mixed chloride and carbonate salt of sevelamer.

11-12. (canceled)

13. The pharmaceutical composition of claim 9, wherein the magnesium compound is magnesium oxide, or a combination of magnesium oxide and magnesium hydroxide.

14-17. (canceled)

18. A pharmaceutical composition, comprising:
a) a crosslinked aliphatic amine polymer or a pharmaceutically acceptable salt thereof; and
b) a pharmaceutically acceptable magnesium compound comprising a magnesium ion, wherein the magnesium compound is entrained within the crosslinked aliphatic amine polymer.

19. (canceled)

20. The pharmaceutical composition of claim 18, wherein the magnesium compound is selected from the group consisting of magnesium oxide, magnesium hydroxide, magnesium carbonate, magnesium formate and a combination thereof.

21. (canceled)

22. The pharmaceutical composition of claim 18, wherein the crosslinked aliphatic amine polymer comprises one or more repeat units represented by a structural formula selected from: 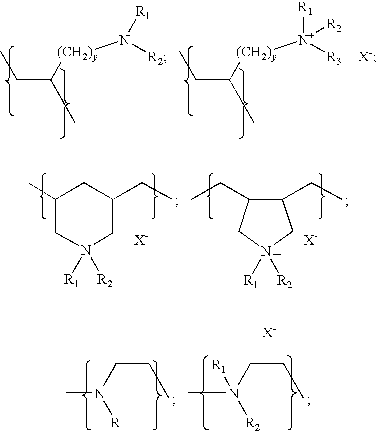
or a salt thereof, wherein:
y and z are independently zero or an integer from one to ten;
R, R₁, R₂ and R₃, independently, are H, a substituted or unsubstituted alkyl group or an aryl group; and
X⁻ is an exchangeable negatively charged counterion.

23-24. (canceled)

25. The pharmaceutical composition of claim 22, wherein the aliphatic amine polymer is a crosslinked polyallylamine.

26. The pharmaceutical composition of claim 25, wherein the crosslinked polyallylamine polymer is sevelamer.

27-29. (canceled)

30. A pharmaceutical composition, comprising:
a) an aliphatic amine polymer or a pharmaceutically acceptable salt thereof; and
b) a pharmaceutically acceptable magnesium compound comprising a magnesium ion, wherein the magnesium compound is selected from the group consisting of magnesium oxide, magnesium hydroxide, magnesium carbonate, magnesium formate and a combination thereof.

31. (canceled)

32. The pharmaceutical composition of claim 30, wherein the aliphatic amine polymer comprises one or more repeat units represented by a structural formula selected from: 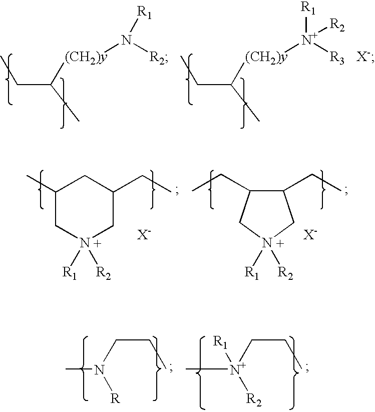
or a salt thereof, wherein:
y and z are independently zero or an integer from one to ten;
R, R₁, R₂ and R₃, independently, are H, a substituted or unsubstituted alkyl group or an aryl group; and
X⁻ is an exchangeable negatively charged counterion.

33-35. (canceled)

36. The pharmaceutical composition of claim 32, wherein the aliphatic amine polymer is a crosslinked polyallylamine.

37. The pharmaceutical composition of claim 36, wherein the polyallylamine polymer is sevelamer.

38-41. (canceled)

42. A pharmaceutical composition, comprising:
a) an aliphatic amine polymer or a pharmaceutically acceptable salt thereof; and
b) a pharmaceutically acceptable magnesium compound comprising a magnesium ion,
wherein the molar ratio of the magnesium ion to amine nitrogen atoms in the aliphatic amine polymer is 0.4-3.0.

43. The pharmaceutical composition of claim 42, wherein the magnesium compound is selected from the group consisting of magnesium oxide, magnesium hydroxide, magnesium carbonate, magnesium formate and a combination thereof.

44. (canceled)

45. The pharmaceutical composition of claim 42, wherein the aliphatic amine polymer comprises one or more repeat units represented by a structural formula selected from: 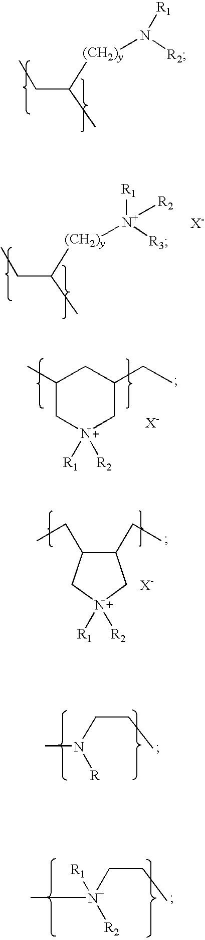
or a salt thereof, wherein:
y and z are independently zero or an integer from one to ten;
R, R₁, R₂ and R₃, independently, are H, a substituted or unsubstituted alkyl group or an aryl group; and
X⁻ is an exchangeable negatively charged counterion.

46-48. (canceled)

49. The pharmaceutical composition of claim 45, wherein the aliphatic amine polymer is a crosslinked polyallylamine.

50. The pharmaceutical composition of claim 49, wherein the polyallylamine polymer is sevelamer.

51-54. (canceled)

55. A method of treating hyperphosphatemia in a patient, comprising the step of administering to the patient an effective amount of a pharmaceutical composition comprising:
a) an aliphatic amine polymer or a pharmaceutically acceptable salt thereof; and
b) a pharmaceutically acceptable magnesium compound comprising a magnesium ion,
wherein the magnesium ion is 5-35% by anhydrous weight of the pharmaceutical composition.

56-70. (canceled)

71. A method of treating hyperphosphatemia in a patient, comprising the step of administering to the patient an effective amount of a pharmaceutical composition comprising:
a) a crosslinked aliphatic amine polymer or a pharmaceutically acceptable salt thereof; and
b) a pharmaceutically acceptable magnesium compound comprising a magnesium ion, wherein the magnesium compound is entrained within the crosslinked aliphatic amine polymer.

72-81. (canceled)

82. A method of treating hyperphosphatemia in a patient, comprising the step of administering to the patient an effective amount of a pharmaceutical composition comprising:
a) an aliphatic amine polymer or a pharmaceutically acceptable salt thereof; and
b) a pharmaceutically acceptable magnesium compound comprising a magnesium ion, wherein the magnesium compound is selected from the group consisting of magnesium oxide, magnesium hydroxide, magnesium carbonate, magnesium formate and a combination thereof.

83-93. (canceled)

94. A method of treating hyperphosphatemia in a patient, comprising the step of administering to the patient an effective amount of a pharmaceutical composition comprising:
a) an aliphatic amine polymer or a pharmaceutically acceptable salt thereof; and
b) a pharmaceutically acceptable magnesium compound comprising a magnesium ion,
wherein the molar ratio of the magnesium ion to amine nitrogen atoms in the aliphatic amine polymer is 0.4-3.0.

95-100. (canceled)
